SEQUENCE LISTING (23 sequences)

SEQ ID NO: 1 - DNA, 4288 bp, Homo sapiens
gcgatcccgggctccccgggctgtgggctacaggcgcagagcgggccaggcgcggagctggcggcagtgacaggaggcgcgaacccgcagcgcttaccgcgcggcgccgcaccatggagcccgccgtgtcgctggccgtgtgcgcgctgctcttcctgctgtgggtgcgcctgaaggggctggagttcgtgctcatccaccagcgctgggtgttcgtgtgcctcttcctcctgccgctctcgcttatcttcgatatctactactacgtgcgcgcctgggtggtgttcaagctcagcagcgctccgcgcctgcacgagcagcgcgtgcgggacatccagaagcaggtgcgggaatggaaggagcagggtagcaagaccttcatgtgcacggggcgccctggctggctcactgtctcactacgtgtcgggaagtacaagaagacacacaaaaacatcatgatcaacctgatggacattctggaagtggacaccaagaaacagattgtccgtgtggagcccttggtgaccatgggccaggtgactgccctgctgacctccattggctggactctccccgtgttgcctgagcttgatgacctcacagtggggggcttgatcatgggcacaggcatcgagtcatcatcccacaagtacggcctgttccaacacatctgcactgcttacgagctggtcctggctgatggcagctttgtgcgatgcactccgtccgaaaactcagacctgttctatgccgtaccctggtcctgtgggacgctgggtttcctggtggccgctgagatccgcatcatccctgccaagaagtacgtcaagctgcgtttcgagccagtgcggggcctggaggctatctgtgccaagttcacccacgagtcccagcggcaggagaaccacttcgtggaagggctgctctactccctggatgaggctgtcattatgacaggggtcatgacagatgaggcagagcccagcaagctgaatagcattggcaattactacaagccgtggttctttaagcatgtggagaactatctgaagacaaaccgagagggcctggagtacattcccttgagacactactaccaccgccacacgcgcagcatcttctgggagctccaggacattatcccctttggcaacaaccccatcttccgctacctctttggctggatggtgcctcccaagatctccctcctgaagctgacccagggtgagaccctgcgcaagctgtacgagcagcaccacgtggtgcaggacatgctggtgcccatgaagtgcctgcagcaggccctgcacaccttccaaaacgacatccacgtctaccccatctggctgtgtccgttcatcctgcccagccagccaggcctagtgcaccccaaaggaaatgaggcagagctctacatcgacattggagcatatggggagccgcgtgtgaaacactttgaagccaggtcctgcatgaggcagctggagaagtttgtccgcagcgtgcatggcttccagatgctgtatgccgactgctacatgaaccgggaggagttctgggagatgtttgatggctccttgtaccacaagctgcgagagaagctgggttgccaggacgccttccccgaggtgtacgacaagatctgcaaggccgccaggcactgagctggagcccgcctggagagacagacacgtgtgagtggtcaggcatcttcccttcactcaagcttggctgctttcctagatccacactttcaaagagaaacccctccagaactcccaccctgacagcccaacaccaccttcctcctggcttccagggggcagcccagtggaatggaaagaatgtgggatttggagtcagacaagcctgagtccagttccccgtttagaactcattagctgtgtgactctgggtgagtcccttaacccctctgagcccgggtctcttcattagttgaaagggatagtaatacctacttgcaggttgttgtcatctgagttgagcactggtcacattgaaggtgctgggtaagtggtagctcttgttgcttcccgttcagcgtcacatctgcagtggagcctgaaaaggctccacattaggtcacctgtgcacagccatggctggaatgatgaaggggatacgctggagttgccctgccatcgcctccatcagccagacgaggtcctcacaggagaaggacagctcttccccaccctgggatctcaggagggcagccacggagtggggaggccccagatgcgctgtgccaaagccaggtccgaggccaaagttctccctgccatccttggtgccgtcctgccccttcctccttcatgcctgggcctgcaggcccaccccagccaccactgagtccactcggagtgccctgtgttcctggagaaggcattccagggttgaatcttgtcccagcctcagcctgggacacctaggtggagagagtggtctccgctctgaattggatccaggggacctgggctcattcttcttggctcaccaaccctgcaggcctcatctttcccaaaacccactttgtcttggtgggagtgggtccgcgctgctctgcagcaggggctggggagtggacagcatcaggtgggaaagtggagtccaccctcatgtttctgtaggattctcaccgtggggctggaagaaaagagcatcgacttgatttctccaaccactcatccctctttttctttcttccaccactccccaccccagctgtagttaatttcagtgccttacaaatcctaagctcagagaaagttccatttccgttccagagggaagggaacctccctaggtccttccctggcttgttataacgcaaagcttggttgtttatgcaactctatcttaagaactgcccagcctcagctgaaaacccgaatctgagaaggaattgcgtcatgtaagggaagctggaattaagggagctgagccagtcatggttgtggcgtgtgagtcaggagacctaggtttcagcccctctctactgtcagcgagctgtgcaacgtgggcaagtcattgtcctctgagctgcagtttcctcatctgtcacatcgctacagacaagacctccctggaacccttctgattgtcttagacactgtggttgcaaaacccacggaaagcctcatttgtgtggaaagtcagaggaaaaatgatccagtggacacttggggattatctgtcattcaagatccttccttcaaccccaaggtcagctcccatctcatttccagaaaggctcatacctggcttgcagggaagcatctgtcttgtcattccaggtgccagaatcctctcagagtcattgaagggtgttcacccatcccacccaaggcttggcacactgccagtgtcttagcagggtcttgtgagggctgggggcatccaggcactcagaaggcaaaggaaccaccctacccatttggcctctggagggggcagaagaaagaaagaaacctcatcctatattttacaaagcatgtgaattctggcattagctctcataggagacccatgtgcttccttgctcagtgcaaaactgatgattctacttgctgtagatgaatggttaacacgagctagttaaacagtgccattgttttgccagtgaagcctccaaccctaagccactgggacggtggccagagatgccagcagcctctgtcgcccttagtcatataaccaaaatccagaccttatccacaacccggggcttggaaaggaaggtattttggaatcacaccctccggttatgttgctccagtaaaatcttgcctggaaagaggcagtcttcttagcatggtgagctgagttcatggcttttttttgtagccagtcctgtccctggccatccatgtgatggttttggatggagttaaacttgatgccagtgggcagtgcatgtggaaagtatcagagtaaggctctcccctccagagccctgagtttcttggctgcatgaaggttttctttagaatcagaattgtagccagtttctttggccagaaggatgaatacttggatattactgaaagggaggggtggagatgggtgtggcagtgtatggtgtgtgatttttattttcttctttggtcatgggggccaaggagaaaggcatgaatcttccctgtcaggctcttacagccacaggcactgtgtctactgtctggaagacatgtccccgtggctgtggggccgctgcttctgtttaaataaaagtggcctggaagctggcaaaaaaaaaaaaaaaaaa

SEQ ID NO: 2 - protein, 516 aa, Homo sapiens
MEPAVSLAVCALLFLLWVRLKGLEFVLIHQRWVFVCLFLLPLSLIFDIYYYVRAWVVFKLSSAPRLHEQRVRDIQKQVREWKEQGSKTFMCTGRPGWLTVSLRVGKYKKTHKNIMINLMDILEVDTKKQIVRVEPLVTMGQVTALLTSIGWTLPVLPELDDLTVGGLIMGTGIESSSHKYGLFQHICTAYELVLADGSFVRCTPSENSDLFYAVPWSCGTLGFLVAAEIRIIPAKKYVKLRFEPVRGLEAICAKFTHESQRQENHFVEGLLYSLDEAVIMTGVMTDEAEPSKLNSIGNYYKPWFFKHVENYLKTNREGLEYIPLRHYYHRHTRSIFWELQDIIPFGNNPIFRYLFGWMVPPKISLLKLTQGETLRKLYEQHHVVQDMLVPMKCLQQALHTFQNDIHVYPIWLCPFILPSQPGLVHPKGNEAELYIDIGAYGEPRVKHFEARSCMRQLEKFVRSVHGFQMLYADCYMNREEFWEMFDGSLYHKLREKLGCQDAFPEVYDKICKAARH

SEQ ID NO: 3 - DNA, 2005 bp, Zea mays
ccgcaggcagcatctgcatctctcgcccacctccgctccgcctactgctgctggtggtagggaggcggagaaggaggcccttgcgcccgcccgccggccggatcagagccgtagcaaccaccacccatggcggacgtgcacgaacctttggtgcgccgtaagaggaagaaggttttggtggactacttggtgaagttccgatggatcctcgtgatcttcgtggtccttcctatttcaactctgatctacttcaacatcttcctgggcgacatgtggtccgccatgaagtcggagaagaagcgccagaagcagcacgacgagaacgtgcagaaggtcgtgaagcggctcaagcagaggaacccgaagaaggacggtcttgtttgcacggccaggaagccctggatcgctgttggcatgcgcaacgtggactacaagcgtgcgaggcatttcgaggtcgacctttcttccttcaggaacatccttgagatcgacaaagagaggatggttgccaaggtcgagccccttgtcaacatgggtcagataaccagagctacctgcccaatgaaccttgcccttgcggtcgtcgccgagctcgacgacctcactgttggtgggctgatcaacggttacggcatcgaggggagctctcacctctatggccttttctccgacacggttgtcgcgatggaggttgttctcgcagatggccgggtcgtcagagccaccaaggacaacgagtactctgaccttttctatggaattccctggtcccagggaacactggggttccttgtctctgcagagatcaagctgatccccatcaaggagtacatgaagctcacctacactccagtcaaggggggtctaaaggagatcgcgcaggcctacgcggattctttcgctccgagggacggtgacccggcaaaggtccctgactttgttgaagggatggtgtacacagagagcgagggtgtcatgatgacgggcgtgtacgcttcgaaagaagaggcgaagaagaagggcaacaagatcaactgcgtggggtggtggtttaagccctggttctaccagcacgctcagacggcgctgaataggggcgagtttgtggagtacatcccgacgagggagtactaccaccggcacacccggtgcctgtactgggaggggaagctgatcctgcccttcggcgaccagttctggttcaggttcctgctgggctggctgatgccaccgaaggtgtccctgctgaaggcgacccagggcgaggctatcaggaactactaccacgacaaccatgtgatccaggacatgctggtgccgctgtacaaggttggggatgcgctggagttcgtgcaccgcgagatggaggtgtatcctctgtggctgtgccctcaccggctgtacaagctgccggtgaagacgatggtgtacccggagcctgggttcgagcaccagcacaggcagggcgacgcgagctacgcacagatgttcacggacgtgggcgtgtactacgcccccggggcggtgctgaggggggaggagttcaacggcgcggaggctgtgcacaggctggagcagtggctgatcgagaaccacagctaccagccgcagtacgcggtgtcggagctgaacgagaaggactcctggcgcatgttcgacgcgtcgcactacgagcactgccgccaaaagtacggggcggtgggcacgttcatgagcgtgtactacaagtccaagaaggggcgcaagacggagaaggaggtgcaggaggcggaggcggccatactggagccggcctacgcggacgaggaggcctaaagctcgtggtcgttttgcttagcccattttaattagaacttgatggatgtagtgtgtgtctgtctgaagtcattttaattagaactcttaaagctcgtggtcggtcggtcagtcagtcagtcattgtcgttgatgtccagcgttgtgttttttttatattctctaatggaatctctcagattgattcggg

SEQ ID NO: 4 - protein, 562 aa, Zea mays
MADVHEPLVRRKRKKVLVDYLVKFRWILVIFVVLPISTLIYFNIFLGDMWSAMKSEKKRQKQHDENVQKVVKRLKQRNPKKDGLVCTARKPWIAVGMRNVDYKRARHFEVDLSSFRNILEIDKERMVAKVEPLVNMGQITRATCPMNLALAVVAELDDLTVGGLINGYGIEGSSHLYGLFSDTVVAMEVVLADGRVVRATKDNEYSDLFYGIPWSQGTLGFLVSAEIKLIPIKEYMKLTYTPVKGGLKEIAQAYADSFAPRDGDPAKVPDFVEGMVYTESEGVMMTGVYASKEEAKKKGNKINCVGWWFKPWFYQHAQTALNRGEFVEYIPTREYYHRHTRCLYWEGKLILPFGDQFWFRFLLGWLMPPKVSLLKATQGEAIRNYYHDNHVIQDMLVPLYKVGDALEFVHREMEVYPLWLCPHRLYKLPVKTMVYPEPGFEHQHRQGDASYAQMFTDVGVYYAPGAVLRGEEFNGAEAVHRLEQWLIENHSYQPQYAVSELNEKDSWRMFDASHYEHCRQKYGAVGTFMSVYYKSKKGRKTEKEVQEAEAAILEPAYADEEA

SEQ ID NO: 5 - DNA, 1963 bp, Arabidopsis thaliana
actttcacatctcatcctctcactctctctctcctttggattcttcagtgaggagaggaaatgtcggatcttcagacaccgcttgtgaggcccaagaggaagaagacttgggttgattactttgtcaagttcagatggatcattgtcatcttcatcgtccttccattctcagccacattctacttcctcatctacctcggggacatgtggtcagagtccaagtcctttgagaaacgtcagaaggaacacgacgagaatgtcaagaaagtcatcaaaaggcttaagggtagggatgcttccaaggacgggcttgtctgcactgctcgtaagccctggatcgctgttggaatgaggaacgttgactacaagagagcccggcatttcgaggttgacttgggggagttccgtaacatccttgagatcaacaaggagaagatgactgctagagtggagcctcttgttaacatgggacagatttcccgtgctaccgtcccaatgaacctgtctctcgctgttgttgctgagcttgatgaccttaccgttggtggacttatcaatggatatggtattgaaggaagctctcacatctacggtttgtttgctgataccgttgaggcttacgagattgttcttgcgggtggagagcttgtccgcgccacaagggataatgagtattctgatctttactacgcaatcccgtggtcgcaaggaactcttggactccttgtagctgctgagatcaggcttattaaagtcaaggagtacatgagactcacttacataccagtcaagggtgatcttcaagccttagctcaaggttacattgattcttttgctcccaaagacggtgacaagtcgaaaatcccggatttcgtcgaaggcatggtttacaatccaacggaaggagtgatgatggttggaacatatgcatctaaagaagaggcaaagaagaaagggaacaaaatcaacaatgtgggatggtggttcaagccgtggttctaccagcacgcgcagaccgccctgaaaaagggacagtttgttgagtacatcccaactcgtgaatactaccacaggcacacaaggtgcttgtactgggaagggaagcttattcttccatttggtgatcagttctggtttaggtacctcttaggttggttgatgcctccaaaggtctctcttcttaaggccactcaaggtgaagctatcaggaactattaccatgatatgcatgttattcaggatatgcttgttcctctttacaaggttggcgatgcactcgaatgggtccaccgcgaaatggaggtgtatccaatttggctttgcccacacaaactcttcaagcagccaatcaaaggccaaatctacccagagccaggcttcgagtacgaaaacagacaaggagacacagaagatgcacagatgtacactgatgttggagtctactacgcacctggctgtgtcctaagaggtgaagagtttgatggatcagaagcagtgcgtaggatggagaaatggctgatagagaaccatggattccagcctcagtacgcggtgtctgagctcgacgagaagagcttctggagaatgtttaatggtgaattgtatgaggagtgccgcaagaagtatagagctattggaacgttcatgagtgtttactacaagtccaagaaaggaaggaagactgagaaagaagttagagaagccgaacaagctcatctcgaaactgcttatgccgaggcagattaagtaataataagccttggtttgatgtaatggctcgggatgattattgttattcctagtagtgtctgttatgttgttgtttcctaatttcagttttagtgtcttgaagactcttgttgttgttgtttgcttcttgttgttgtttgactcattctggtttgagtggattttagagtttaaaacttttggatttgcctatgaaattgaaggtttccggttt

SEQ ID NO: 6 - protein, 561 aa, Arabidopsis thaliana
MSDLQTPLVRPKRKKTWVDYFVKFRWIIVIFIVLPFSATFYFLIYLGDMWSESKSFEKRQKEHDENVKKVIKRLKGRDASKDGLVCTARKPWIAVGMRNVDYKRARHFEVDLGEFRNILEINKEKMTARVEPLVNMGQISRATVPMNLSLAVVAELDDLTVGGLINGYGIEGSSHIYGLFADTVEAYEIVLAGGELVRATRDNEYSDLYYAIPWSQGTLGLLVAAEIRLIKVKEYMRLTYIPVKGDLQALAQGYIDSFAPKDGDKSKIPDFVEGMVYNPTEGVMMVGTYASKEEAKKKGNKINNVGWWFKPWFYQHAQTALKKGQFVEYIPTREYYHRHTRCLYWEGKLILPFGDQFWFRYLLGWLMPPKVSLLKATQGEAIRNYYHDMHVIQDMLVPLYKVGDALEWVHREMEVYPIWLCPHKLFKQPIKGQIYPEPGFEYENRQGDTEDAQMYTDVGVYYAPGCVLRGEEFDGSEAVRRMEKWLIENHGFQPQYAVSELDEKSFWRMFNGELYEECRKKYRAIGTFMSVYYKSKKGRKTEKEVREAEQAHLETAYAEAD

SEQ ID NO: 7 - DNA, 1686 bp, Oryza sativa
atggcagatctgcaggagcccctcgttcgtccgaagaggaagaaggttttggtggactacttggtaaagttccgatggattctggtgatctttgtggtgctccccatttccgctctgatctacttcaatatctatttgggcgatgtctggtctgccatgaaatctgagaaacgtcgccagaaggaacatgatgacaatgtgcaaaaagttgtgaagcggctcaagcagaggaacccaaagaaggatggccttgtttgcacagctaggaagccctggattgctgttggcatgcgcaatgtagactacaagcgtgctaggcattttgaggttgacctttccgccttcaggaacattcttgagattgacagagagagaatggttgccaaggttgagcctcttgtcaacatgggccagataaccagagctacatgcccaatgaaccttgcccttgcagttgttgctgagcttgatgaccttactgttgggggactgatcaatgggtatggtattgaagggagctctcacctctatggtcttttctctgacactgttgtcgccgtggaagttgttcttgcagacggtcgagttgttagagccactaaggataatgagtactctgaccttttctatggcattccctggtcccagggaacacttgggtttcttgtttccgctgagatcaaactcattcccatcaaggaatacatgaggctcacatatactccagttaaagggtcactgaaggagatagcacaaggttattgtgattcgtttgcaccacgagatggtgatcctgcaaaggtcccagacttcgttgagggaatggtgtacacagaaaatgagggtgtcatgatgactggtgtttatgcttccaaagaagaggcaaagaagaagggcaataagatcaactgtgtcgggtggtggttcaagccttggttttaccaacatgctcagacagcactcaagaagggtgagtttgtggagtacattccaacaagagagtactaccaccgtcacacccggtgtctgtactgggaggggaagctgatcttgccattcggcgaccaattctggttcaggttcctcttgggctggctgatgccaccaaaggtgtctctgctcaaggccacacagggtgaatctatcaggaattactaccatgacaaccatgtgattcaagacatgctggttcccttgtacaaagttggagatgctcttgagtttgttcacaaggaaatggaggtttatccactgtggctgtgcccgcaccggctctacaagctccctgtgaaaaccatggtgtacccagagcctggctttgagcaccaccacaggcaaggtgacactagctatgcccagatgttcaccgatgttggtgtgtactatgctcctggtgctgtcctgaggggcgaggagttcaatggcgctctagctgtccacaggctggagcagtggctgattgagaaccacagctaccagccacagtacgctgtatctgagctcaacgagaaggacttctggaggatgtttgatgcttctcactacgagcattgccgccaaaagtatggtgccgtcggtacctttatgagcgtctactacaagtccaagaagggaaggaagactgagaaggaggtgcaggaagccgaggccgccatcctcgagccagcctacgctgatgaggcgtaa

SEQ ID NO: 8 - protein, 561 aa, Oryza sativa
MADLQEPLVRPKRKKVLVDYLVKFRWILVIFVVLPISALIYFNIYLGDVWSAMKSEKRRQKEHDDNVQKVVKRLKQRNPKKDGLVCTARKPWIAVGMRNVDYKRARHFEVDLSAFRNILEIDRERMVAKVEPLVNMGQITRATCPMNLALAVVAELDDLTVGGLINGYGIEGSSHLYGLFSDTVVAVEVVLADGRVVRATKDNEYSDLFYGIPWSQGTLGFLVSAEIKLIPIKEYMRLTYTPVKGSLKEIAQGYCDSFAPRDGDPAKVPDFVEGMVYTENEGVMMTGVYASKEEAKKKGNKINCVGWWFKPWFYQHAQTALKKGEFVEYIPTREYYHRHTRCLYWEGKLILPFGDQFWFRFLLGWLMPPKVSLLKATQGESIRNYYHDNHVIQDMLVPLYKVGDALEFVHKEMEVYPLWLCPHRLYKLPVKTMVYPEPGFEHHHRQGDTSYAQMFTDVGVYYAPGAVLRGEEFNGALAVHRLEQWLIENHSYQPQYAVSELNEKDFWRMFDASHYEHCRQKYGAVGTFMSVYYKSKKGRKTEKEVQEAEAAILEPAYADEA

SEQ ID NO: 9 - protein, 561 aa, Arabidopsis thaliana
MSDLQTPLVRPKRKKTWVDYFVKFRWIIVIFIVLPFSATFYFLIYLGDMWSESKSFEKRQKEHDENVKKVIKRLKGRDASKDGLVCTARKPWIAVGMRNVDYKRARHFEVDLGEFRNILEINKEKMTARVEPLVNMGQISRATVPMNLSLAVVAELDDLTVGGLINGYGIEGSSHIYGLFADTVEAYEIVLAGGELVRATRDNEYSDLYYAIPWSQGTLGLLVAAEIRLIKVKEYMRLTYIPVKGDLQALAQGYIDSFAPKDGDKSKIPDFVEGMVYNPTEGVMMVGTYASKEEAKKKGNKINNVGWWFKPWFYQHAQTALKKGQFVEYIPTREYYHRHTRCLYWEGKLILPFGDQFWFRYLLGWLMPPKVSLLKATQGEAIRNYYHDMHVIQDMLVPLYKVGDALEWVHREMEVYPIWLCPHKLFKQPIKGQIYPEPGFEYENRQGDTEDAQMYTDVGVYYAPGCVLRGEEFDGSEAVRRMEKWLIENHGFQPQYAVSELDEKSFWRMFNGELYEECRKKYRAIGTFMSDYYKSKKGRKTEKEVREAEQAHLETAYAEAD

SEQ ID NO: 10 - protein, 561 aa, Arabidopsis thaliana
MSDLQTPLVRPKRKKTWVDYFVKFRWIIVIFIVLPFSATFYFLIYLGDMWSESKSFEKRQKEHDENVKKVIKRLKGRDASKDGLVCTARKPWIAVGMRNVDYKRARHFEVDLGEFRNILEINKEKMTARVEPLVNMGQISRATVPMNLSLAVVAELDDLTVGGLINGYGIEGSSHIYGLFADTVEAYEIVLAGGELVRATRDNEYSDLYYAIPWSQGTLGLLVAAEIRLIKVKEYMRLTYIPVKGDLQALAQGYIDSFAPKDGDKSKIPDFVEGMVYNPTEGVMMVGTYASKEEAKKKGNKINNVGWWFKPWFYQHAQTALKKGQFVEYIPTREYYHRHTRCLYWEGKLILPFGDQFWFRYLLGWLMPPKVSLLKATQGEAIRNYYHDMHVIQDMLVPLYKVGDALEWVHREMEVYPIWLCPHKLFKQPIKGQIYPEPGFEYENRQGDTEDAQMYTDVGVYYAPGCVLRGEEFDGSEAVRRMEKWLIENHGFQPQYAVSELDEKSFWRMFNGELYEECRKDGDAIGTFMSVYYKSKKGRKTEKEVREAEQAHLETAYAEAD

SEQ ID NO: 11 - protein, 539 aa, Arabidopsis thaliana
MSDLQTPLVRPKRKKTWVDYFVKFRWIIVIFIVLPFSATFYFLIYLGDMWSESKSFEKRQKEHDENVKKVIKRLKGRDASKDGLVCTARKPWIAVGMRNVDYKRARHFEVDLGEFRNILEINKEKMTARVEPLVNMGQISRATVPMNLSLAVVAELDDLTVGGLINGYGIEGSSHIYGLFADTVEAYEIVLAGGELVRATRDNEYSDLYYAIPWSQGTLGLLVAAEIRLIKVKEYMRLTYIPVKGDLQALAQGYIDSFAPKDGDKSKIPDFVEGMVYNPTEGVMMVGTYASKEEAKKKGNKINNVGWWFKPWFYQHAQTALKKGQFVEYIPTREYYHRHTRCLYWEGKLILPFGDQFWFRYLLGWLMPPKVSLLKATQGEAIRNYYHDMHVIQDMLVPLYKVGDALEWVHREMEVYPIWLCPHKLFKQPIKGQIYPEPGFEYENRQGDTEDAQMYTDVGVYYAPGCVLRGEEFDGSEAVRRMEKWLIENHGFQPQYAVSELDEKSFWRMFNGELYEEKTEKEVREAEQAHLETAYAEAD

SEQ ID NO: 12 - protein, 22 aa, Arabidopsis thaliana
CRKKYRAIGTFMSDYYKSKKGR

SEQ ID NO: 13 - protein, 22 aa, Arabidopsis thaliana
CRKDGDAIGTFMSVYYKSKKGR

SEQ ID NO: 14 - protein, 472 aa, Arabidopsis thaliana
MAFTAFLLLLSSIAAGFLLLLRRTRYRRMGLPPGSLGLPLIGETFQLIGAYKTENPEPFIDERVARYGSVFMTHLFGEPTIFSADPETNRFVLQNEGKLFECSYPASICNLLGKHSLLLMKGSLHKRMHSLTMSFANSSIIKDHLMLDIDRLVRFNLDSWSSRVLLMEEAKKITFELTVKQLMSFDPGEWSESLRKEYLLVIEGFFSLPLPLFSTTYRKAIQARRKVAEALTVVVMKRREEEEEGAERKKDMLAALLAADDGFSDEEIVDFLVALLVAGYETTSTIMTLAVKFLTETPLALAQLKEEHEKIRAMKSDSYSLEWSDYKSMPFTQCVVNETLRVANIIGGVFRRAMTDVEIKGYKIPKGWKVFSSFRAVHLDPNHFKDARTFNPWRWQSNSVTTGPSNVFTPFGGGPRLCPGYELARVALSVFLHRLVTGFSWVPAEQDKLVFFPTTRTQKRYPIFVKRRDFAT

SEQ ID NO: 15 - protein, 513 aa, Arabidopsis thaliana
MFETEHHTLLPLLLLPSLLSLLLFLILLKRRNRKTRFNLPPGKSGWPFLGETIGYLKPYTATTLGDFMQQHVSKYGKIYRSNLFGEPTIVSADAGLNRFILQNEGRLFECSYPRSIGGILGKWSMLVLVGDMHRDMRSISLNFLSHARLRTILLKDVERHTLFVLDSWQQNSIFSAQDEAKKFTFNLMAKHIMSMDPGEEETEQLKKEYVTFMKGVVSAPLNLPGTAYHKALQSRATILKFIERKMEERKLDIKEEDQEEEEVKTEDEAEMSKSDHVRKQRTDDDLLGWVLKHSNLSTEQILDLILSLLFAGHETSSVAIALAIFFLQACPKAVEELREEHLEIARAKKELGESELNWDDYKKMDFTQCVINETLRLGNVVRFLHRKALKDVRYKGYDIPSGWKVLPVISAVHLDNSRYDQPNLFNPWRWQQQNNGASSSGSGSFSTWGNNYMPFGGGPRLCAGSELAKLEMAVFIHHLVLKFNWELAEDDQPFAFPFVDFPNGLPIRVSRIL

SEQ ID NO: 16 - protein, 432 aa, Arabidopsis thaliana
MAETVHSPIVTYASMLSLLAFCPPFVILLWYTMVHQDGSVTQTFGFFWENGVQGLINIWPRPTLIAWKIIFCYGAFEAILQLLLPGKRVEGPISPAGNRPVYKANGLAAYFVTLATYLGLWWFGIFNPAIVYDHLGEIFSALIFGSFIFCVLLYIKGHVAPSSSDSGSCGNLIIDFYWGMELYPRIGKSFDIKVFTNCRFGMMSWAVLAVTYCIKQYEINGKVSDSMLVNTILMLVYVTKFFWWEAGYWNTMDIAHDRAGFYICWGCLVWVPSVYTSPGMYLVNHPVELGTQLAIYILVAGILCIYINYDCDRQRQEFRRTNGKCLVWGRAPSKIVASYTTTSGETKTSLLLTSGWWGLARHFHYVPEILSAFFWTVPALFDNFLAYFYVIFLTLLLFDRAKRDDDRCRSKYGKYWKLYCEKVKYRIIPGIY

SEQ ID NO: 17 - protein, 27 aa, Arabidopsis thaliana
PLFSTTYRKAIQARRKVAEALTVVVMK

SEQ ID NO: 18 - protein, 25 aa, Arabidopsis thaliana
PSLLSLLLFLILLKRRNRKTRFNLP

SEQ ID NO: 19 - protein, 24 aa, Arabidopsis thaliana
TLRLGNVVRFLHRKALKDVRYKGY

SEQ ID NO: 20 - protein, 25 aa, Arabidopsis thaliana
CRSKYGKYWKLYCEKVKYRIIPGIY

SEQ ID NO: 21 - protein, 23 aa, Arabidopsis thaliana
CRKKYRAIGTFMSVYYYKSKKGR

SEQ ID NO: 22 - protein, 25 aa, Arabidopsis thaliana
PSLLSDLLFLILLKRRNRKTRFNLP

SEQ ID NO: 23 - protein, 25 aa, Arabidopsis thaliana
PSLLSLLLFLILVKRRNRKTRFNLP

(19) United States
(12) Patent Application Publication
(10) Pub. No.: US 2007/0089183 A1
(43) Pub. Date: Apr. 19, 2007

(54) Size and/or growth engineering by modulation of the interaction between calmodulin, and brassinosteroid biosynthetic enzymes and orthologs therof

(75) Inventors: Bachettira W. Poovaiah, Pullman, WA (US); Liqun Du, Pullman, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Washington State University, Pullman, WA (US)

(21) Appl. No.: 11/518,331

(22) Filed: Sep. 6, 2006

Related U.S. Application Data
(60) Provisional application No. 60/714,313, filed on Sep. 6, 2005.

(51) Int. Cl. A01H 5/00; C07H 21/04; C12N 9/10; C12N 15/82; C12N 5/04

(52) U.S. Cl. 800/278; 800/295; 435/468; 435/419; 435/193; 536/23.2

(57) ABSTRACT

Particular aspects show that DWF1 is a Ca²⁺/calmodulin-binding protein, and this binding is critical for function. Molecular/genetic analysis using site-directed and deletion mutants revealed that loss of calmodulin binding abolished the function of DWF1 in planta, whereas partial loss of calmodulin binding resulted in a partial dwarf phenotype in complementation studies, providing direct proof that Ca²⁺/calmodulin-mediated signalling has a critical role in controlling the DWF1 function. Furthermore, DWF1 orthologues from other plants have a similar Ca²⁺/calmodulin-binding domain, indicating that Ca²⁺/calmodulin regulation of DWF1 and its homologues is common, and broadly applicable in plants. Methods for generating size-engineered crops are provided by altering the Ca²⁺/calmodulin-binding property of their DWF1 orthologues. Likewise, according to additional aspects, DWF4, DWF5 and CPD were also shown to be Ca²⁺/calmodulin-binding proteins, and can likewise be used for generating size-engineered crops by altering the Ca²⁺/calmodulin-binding property of their orthologues in a variety of plants, including crop plants.

### CROSS-REFERENCE TO RELATED APPLICATIONS

This application claims the benefit of priority to U.S. Provsional Patent Application Ser. No. 60/714,313, filed 6 Sep. 2005 and entitled “SIZE AND/OR GROWTH ENGINEERING BY MODULATION OF THE INTERACTION BETWEEN CALMODULIN, AND BRASSINOSTEROID BIOSYNTHETIC ENZYMES AND ORTHOLOGS THEREOF,” which is incorporated by reference herein in its entirety.

### FEDERAL FUNDING

This work was partially supported by grants from the National Science Foundation (MCB0424898) and the United States Department of Agriculture (2005-35100-15985), and the United States Government may therefore having certain rights.

### FIELD OF THE INVENTION

Aspects of the present invention relate to the production of engineered (e.g., size and/or productivity engineered) life forms, and in particular aspects provide compositions and methods that are broadly applicable to engineered (e.g., size and/or productivity engineered) plants.

### BACKGROUND

The “Green Revolution” is credited with increasing crop yield on a global scale, and was fueled by the introduction of semi-dwarf features into cereal crop plants. These semi-dwarf cultivars use less water and are more resistant to wind and rain damage and thus yield more grain when fertilized. However, development of such cultivars by traditional methods is both time-consuming and expensive, and there is a pronounced need in the art for novel and efficient approaches to produce semi-dwarf or size-engineered plants and crop breeders, particularly approaches applicable to a wide range of plants, and particularly approaches that allow for fine-tuning the characteristics (e.g., growth, size, productivity, fertility, etc.) of the engineered plants.

Brassinosteroids are plant-specific steroid hormones^{1,2} that have an important role in coupling environmental factors, especially light, with plant growth and development³. The importance of brassinosteroids in plant growth and development is well demonstrated by Arabidopsis mutants losing their ability to synthesize or perceive brassinosteroids. These mutants usually show a characteristic pleiotropic phenotype including dwarfism with dark-green colour, photomorphogenesis in the dark, delayed senescence, and reduced apical dominance and fertility^{1,2}. The understanding of the brassinosteroid biosynthetic pathway, which ends in the production of brassinolide, has greatly expedited brassinosteroid signalling research^{8,9}. The endogenous homeostasis of this class of powerful growth regulators is delicately tuned by feedback controls at several steps in the brassinosteroid biosynthetic pathway, such as DWF4, CPD and BR6OX, through the involvement of brassinosteroid perception and connection of the downstream pathway to transcriptional regulation^{8,9}. Recent reports have revealed some exciting results concerning brassinosteroid signal perception and signal relay to the nucleus, and key components of brassinosteroid signalling, such as BRI1 (ref. 10), BAK1 (refs 11, 12), BIN2 (ref. 13) and BES1/BZR1 (refs 1, 2), have been identified and characterized. However, how brassinosteroid action is altered by environmental stimuli remains largely unknown. Recently Pra2, a light-responsive small G protein, was found to interact and regulate DDWF1, a cytochrome P450 from pea that converts typhasterol to castasterone¹⁴. This is one possible way for light to regulate endogenous brassinosteroid levels. Other environmental signalling events may crosstalk with brassinosteroid signalling and modulate the endogenous brassinosteroid homeostasis; however, little is known about these events⁹.

Ca²⁺/calmodulin has an essential role in sensing and transducing environmental stimuli^{4,5}.

Arabidopsis DWARF1 (DWF1) is responsible for an early step in brassinosteroid biosynthesis that converts 24-methylenecholesterol to campesterol^{6,7}. DWF4, DWF5 and CPD are three other important enzymes for brassinosteroid biosynthesis.

### SUMMARY OF PARTICULAR ASPECTS

Calcium/calmodulin-mediated signaling is known to be involved in plant growth and development, but the nature and mechanism of such involvement is unknown. Likewise, brassinosteroids (plant growth hormones) are known to play an important role in coupling environmental factors such as light with plant growth and development, but the nature and mechanism of such coupling is unknown.

Particular aspects show that DWF1 is a Ca²⁺/calmodulin-binding protein, and this binding is critical for its function. Molecular genetic analysis using site-directed and deletion mutants revealed that loss of calmodulin binding totally abolished the function of DWF1 in planta, whereas partial loss of calmodulin binding resulted in a partial dwarf phenotype in complementation studies. These results provide direct proof that Ca²⁺/calmodulin-mediated signalling has a critical role in controlling the function of DWF1. Furthermore, DWF1 orthologues from other plants have a similar Ca²⁺/calmodulin-binding domain, indicating that Ca²⁺/calmodulin regulation of DWF1 and its homologues is common in plants. These results provide methods for generating size-engineered crops by altering the Ca²⁺/calmodulin-binding property of their DWF1 orthologues. Likewise, according to additional inventive aspects, DWF4, DWF5 and CPD were also shown to be Ca²⁺/calmodulin-binding proteins, and can be used to provide methods for generating size-engineered crops by altering the Ca²⁺/calmodulin-binding property of their orthologues in a variety of plants, including crop plants.

Particular aspects provide a new biotechnological approach to produce semi-dwarf or size-engineered plants. In certain aspects, the approach is applicable to a wide range of plants, including crop plants (e.g., Hops, and other crop plants), and is valuable for producing crop breeders. Particular aspects show that the calcium messenger system (e.g., CA²⁺/Calmodulin) is critical for brassinosteroid biosynthesis and plant growth.

In specific aspects, a molecular genetic approach was used to change the calmodulin-binding property of a key enzyme (DWARF1 (DWF1), or orthologs thereof) in the biosynthesis of brassinosteroids, and demonstrate that this binding is critical for its function. In additional aspects, plant height was controlled by creating mutant plants carrying the altered DWF1 genes.

In yet further inventive aspects, DWF4, DWF5, and CPD (three other important enzymes for brassinosteroid biosynthesis) are also demonstrated to be CA²⁺/Calmodulin binding proteins, and provide for additional methods and compositions for plant engineering by altering the CA²⁺/Calmodulin binding properties thereof.

Preferred aspects provide novel methods and compositions for producing size-engineered plants, and further provide the resulting engineered plants themselves.

### BRIEF DESCRIPTION OF THE DRAWINGS

FIG. 1 shows, according to particular aspects of the present invention, the Ca²⁺/CaM-binding property of DWF1. Panel A shows a ³⁵S-CaM-binding assay. Total proteins from E. coli expressing pScreen-1b (lane 1) or expressing pScreen-1b-517-561 (lane 2) were stained (left panel) or bound to ³⁵S-CaM in the presence of Ca²⁺ (middle panel) or EGTA (right panel), a Ca²⁺ chelator. Panel B shows co-immunoprecipitation of Flag-tagged DWF1 and CaM. Total proteins from plants expressing the Flag-tagged DWF1 (DWF1-Flag) or wild-type plants (WT) were incubated with antibodies against full-length human CaM1 (anti-CaM1), AtBET10 (anti-AtBET10) or non-immune serum (Non-im.); immunocomplexes were separated on SDS-PAGE and detected with anti-Flag M2 monoclonal antibody. Panel D shows mapping of the CaMBD of DWF1. Total proteins from E. coli expressing pET32b (lane 1), pET32b-1-349 (lane 2), pET32b-348-518 (lane 3), pScreen-1b (lane 4) or pScreen-1b-517-561 (lane 5) were stained (left) or were bound to ³⁵S-CaM (right). Panel D shows helical wheel projection of amino acids 518-535 from DWF1 CaMBD (518-539). Positively charged residues are marked with a plus sign, whereas hydrophobic residues are boxed; amino acids 518-535 exhibit a typical conserved CaM-binding secondary structure: a typical amphiphilic a-helix with a positively charged face on one side and a predominantly hydrophobic face on the other side. Panel E shows gel mobility shift assay of synthetic DWF1 CaMBD: the gel mobility shift was tested using 120 pmol potato CaM6 and increasing amounts of synthetic peptide (peptide/CaM molar ratios are indicated) in the presence of 1 mM Ca²⁺ (top) or 5 mM EGTA (bottom). CaM and the peptide-CaM complex is indicated. Panel F shows interaction between dansyl-CaM and the CaM-binding peptide (CRKKYRAIGTFMSVYYKSKKGR) (SEQ ID NO:21) of DWF1, as revealed by fluorescence titration.

FIG. 2 shows, according to particular aspects of the present invention, that the DWF1 null mutant line Salk_{—}006932 can be rescued by 35S::DWF1. Panel A shows twenty-four-day-old plants (wild-type, DD; heterozygous dwf1 mutant, Dd; homozygous dwf1 mutant, dd) with Columbia background are shown. Panel B shows molecular evidence supporting the genotype of plants in Panel A. Top panel of Panel B: positive PCR1 (Lba-1 and D-2 primers, see supplemental figure for primer positions) indicates the existence of insertion; negative PCR2 (D-5u and D-2 primers) indicates double insertions in both copies of endogenous DWF1. Middle panel of Panel B: northern blot probed with DWF1 coding sequence (cds); ethidium bromide (EB) staining was used as a loading control. Bottom panel of Panel B: RT-PCR showing the expression of DWF1 with primer D-5u and D-2; expression of actin 3 (U39480) was used as a control. Panel C shows twenty-four-day-old plants: wild-type, W; dwarf, d; and eight individual T1 mutant plants complemented with the 35S::DWF1 construct. Panel D shows molecular evidence supporting the genotype of plants in Panel C. Top panel of Panel D: PCR1 and PCR2 (see Panel B); positive PCR3 (D-1 and D-2 primers) indicates the existence of a complementary 35S::DWF1. Bottom panel of Panel D: northern blot probed with DWF1 cds (detects both transgenic and endogenous transcription) and 3′ end untranslated sequence of DWF1 (3uts; detects endogenous transcription only); EB staining was used as a loading control.

FIG. 3 shows, according to aspects of the present invention, that loss of CaM binding nullifies DWF1 function. Panel A shows a ³⁵S-CaM-binding assay of purified recombinant protein (2 μg per lane) covering amino acid 517 to the stop codon of DWF1(WT), DWF1(V531D), DWF1(KYR521-523DGD) and DWF1(Δ518-539) (W, M1, M2 and M3, respectively). Panel B shows twenty-four-day-old plants: two plants are shown for each line. Panel C shows forty-eight-day-old plants of wild-type (W), dwarf (d) and dwarf mutants complemented with 35S::DWF1(WT) (cW line 3 in FIG. 2C), DWF1(V53]D) (cM1), DWF1(KYR521-523DGD) (cM2) and DWF1(Δ518-539) (cM3) constructs. Panel D shows Northern blot hybridized to cds and 3uts probes of DWF1, demonstrating the genotypes of these plants. Panel E shows GC-MS analysis showing the endogenous 24-methylenecholesterol/campesterol and sitosterollisoficosterol levels sitosterol, isofucosterol and unresolved 24-methylenecholesterol/campesterol peaks are indicated with retention times (min) shown by values in filled boxes, open boxes and without boxes, respectively. The wild-type plants (W) and DWF1 complemented dwf1 mutant plants (cW) had a very similar sitosterol/isofucosterol pattern with a large sitosterol peak and a very small isofucosterol peak. The dwf1 mutant (d) as well as cM2 and cM3 had large isofucosterol peaks and no sitosterol peak; cM1 had a large isofucosterol peak and a faint sitosterol peak. Panel F shows expression levels of brassinosteroid-regulated DWF4 and CPD (brassinosteroid markers) reflecting endogenous brassinosteroid levels.

FIG. 4 shows, according to additional aspects of the present invention, that the C-terminal CaMBD is unique to plant orthologues of DWF1. Panel A shows a comparison of the C terminus of DWF1 orthologues from Arabidopsis (At), pea (Ps), rice (Os), human (Hs), mouse (Mm) and C. elegans (Ce). The CaMBD of each plant orthologue is underlined. Panel B shows a ³⁵S-CaM-binding assay of DWF1 orthologues. Total proteins from E. coli expressing pScreen-1b (lane 1) or pScreen-1b carrying the boxed segment in a of DWF1 orthologues from Arabidopsis (lane 2), pea (3), rice (4), human (5) and mouse (6) were stained (left) or bound to ³⁵S-CaM in the presence of Ca²⁺ (right).

FIG. 5 shows, according to additional aspects of the present invention, a gel mobility shift assay showing several additional BR biosynthetic proteins (DWF4 (SEQ ID NO:15) DWF5 (SEQ ID NO:16) and CPD) (SEQ ID NO:14) are Ca²⁺/CaM-binding proteins. PCM1: potato calmodulin 1; PCM6: potato calmodulin 6. Sequences of respective CaMBD are also shown: N-DWF4 (SEQ ID NO:18); C-DWF4 (SEQ ID NO:19); CPD (SEQ ID NO:17); and DWF5 (SEQ ID NO:20).

FIG. 6 shows a schematic illustration of endogenous DWFV1 and its complementary constructs. Panel A shows endogenous DWF1, T-DNA insert in Salk_{—}006932 line, primers used for checking T-DNA insert, knockout status, and transgene are indicated, D1 match from 1 (ATG) to 20 bp; D2, 1056 to 1036; D5u −24 to −2 of DWF1 gene. LBa1 sequence was previously described²³. Panel B shows complementary constructs of DWF1 and its mutants. Primers used for checking transgene are indicated, and the CaMBD is enlarged, nucleotide and amino acid sequences of wildtype CaMBD are in blue, the mutated positions are in red and underlined, deleted parts are joined with a bent line.

FIG. 7, shows a scheme of the Sterol and brassinosteroid biosynthetic pathway leading to the production of brassinolide. This illustration depicts key enzymes involved in the BR biosynthetic pathway and was created based on published results. Particular enzymes displayed are steps selected for modulation by the presently disclosed methods.

FIG. 8 shows, according to additional aspects, plants of altered sizes. FIG. 8A shows DWF1 function and plant growth, wildtype (1); dwf1 null mutant (2); dwf1 mutant complemented with wildtype (3) and mutated DWF1 (4, 5, 6; the three mutants are described herein). FIG. 8B shows DWF4 function and plant growth, wildtype (1), dwf4 null mutant (2), dwf4 mutant complemented with mutated DWF4 (3) (containing a L to D change at aa 21 within the N-terminal CaMBD of DWF4) and DWF4 over-expressing plants (4 (containing a L to V change at aa 28 within the N-termainl CaMBD of DWF4), 5). FIG. 8C shows a schematic presentation of constructs expressing wildtype, and mutated versions of DWF4; showing the CaMBD of DWF4 variants L21D (SEQ ID NO:22) and L28V (SEQ ID NO:23).

### DETAILED DESCRIPTION OF EXEMPLARY EMBODIMENTS

Particular aspects show that DWF1 is a Ca²⁺/calmodulin-binding protein, and that this binding is critical for DWF1 function. Molecular/genetic analysis using site-directed and deletion mutants revealed that loss of calmodulin binding abolished the function of DWF1 in planta, whereas partial loss of calmodulin binding resulted in a partial dwarf phenotype in complementation studies, providing direct proof that Ca²⁺/calmodulin-mediated signalling has a critical role in controlling the DWF1 function. Furthermore, DWF1 orthologues from other plants have a similar Ca²⁺/calmodulin-binding domain, indicating that Ca²⁺/calmodulin regulation of DWF1 and its homologues is common, and broadly applicable in plants. Methods for generating size-engineered crops are provided by altering the Ca²⁺/calmodulin-binding property of their DWF1 orthologues. Likewise, according to additional aspects, DWF4, DWF5 and CPD, other brassinosteroid biosyntetic enzymes, were also shown to be Ca²⁺/calmodulin-binding proteins, and can likewise be used for generating size-engineered crops by altering the Ca²⁺/calmodulin-binding property of their orthologues in a variety of plants, including crop plants.

As disclosed herein, altering the Ca²⁺/calmodulin-binding property comprises introducing at least one amino acid sequence alteration into the amino acid sequence of at least one brassinosteroid biosyntetic enzyme, wherein the at least one amino acid sequence alteration modifies a calmodulin-binding activity of the at least one enzyme, wherein, for example in particular aspects, the at least one plant characteristic is modified.

In certain embodiments, the at least one amino acid sequence alteration comprises an amino acid substitution, deletion or insertion. In particular aspects, the at least one amino acid sequence alteration comprises an amino acid substitution. In certain aspects, the at least one amino acid sequence alteration comprises a non-conservative amino acid substitution.

Recombinant Techniques:

Homologous sequences are found when there is an identity of sequence and may be determined upon comparison of sequence information, nucleic acid or amino acid, or through hybridization reactions between a known and a candidate source. Conservative changes (see in more detail below), such as Glu/Asp, Val/Ile, Ser/Thr, Arg/Lys and Gln/Asn may also be considered in determining sequence homology. Typically, a lengthy nucleic acid sequence may show as little as 50-60%, 60%-70%, 80% to 80% sequence identity, and more preferably at least about 70% or about 80% sequence identity, between the target sequence and the given plant sequence of interest excluding any deletions which may be present, and still be considered related. Amino acid sequences are considered homologous by as little as 25% sequence identity between the two complete mature proteins. (see generally, Doolittle, R. F., OF URFS and ORFS (University Science Books, California, 1986). Nucleic acid sequences which encode the disclosed plant enzymes may be used in various constructs, for example, as probes to obtain further sequences. Alternatively, these sequences may be used in conjunction with appropriate regulatory sequences to increase levels of the respective enzymes (or mutant enzymes) of interest in a host cell for recovery or study of the enzyme in vitro or in vivo or to decrease levels or activities of other enzymes of interest for some applications when the host cell is a plant entity, including plant cells, plant parts (including but not limited to seeds, cuttings or tissues) and plants. A nucleic acid sequence encoding a novel plant enzyme disclosed herein may include genomic, cDNA or mRNA sequence. “Encoding” means that the sequence corresponds to a particular amino acid sequence either in a sense or anti-sense orientation. By “extrachromosomal” is meant that the sequence is outside of the plant genome of which it is naturally associated. “Recombinant” means that the sequence contains a genetically engineered modification through manipulation via mutagenesis, restriction enzymes, and the like. A cDNA sequence may or may not contain pre-processing sequences, such as transit peptide sequences. Transit peptide sequences facilitate the delivery of the protein to a given organelle and are cleaved from the amino acid moiety upon entry into the organelle, releasing the “mature” sequence. The use of the precursor plant DNA sequence is preferred in plant cell expression cassettes.

Once the desired nucleic acid sequence is obtained, it may be manipulated in a variety of ways. Where the sequence involves non-coding flanking regions, the flanking regions may be subjected to resection, mutagenesis, etc. Thus, transitions, transversions, deletions, and insertions may be performed on the naturally occurring sequence. In addition, all or part of the sequence may be synthesized. In the structural gene, one or more codons may be modified to provide for a modified amino acid sequence, or one or more codon mutations may be introduced to provide for a convenient restriction site or other purpose involved with construction or expression. The structural gene may be further modified by employing synthetic adapters, linkers to introduce one or more convenient restriction sites, or the like.

Generally, the constructs will involve regulatory regions functional in plants, plant tissues (e.g., seed tissue) or other organisms (e.g., yeast) which provide for modified production of plant enzymes, and for example, growth engineering. The open reading frame, coding for the plant enzyme or functional fragments thereof will be joined at its 5′ end to a transcription initiation regulatory region such as, for example, the wild-type sequence naturally found 5′ upstream to the respective structural gene. Numerous other transcription initiation regions are available which provide for a wide variety of constitutive or regulatable, e.g., inducible, transcription of the structural gene functions. Among transcriptional initiation regions used for plants are such regions associated with the structural genes such as for nopaline and mannopine synthases, or with napin, ACP promoters and the like. The transcription/translation initiation regions corresponding to such structural genes are found immediately 5′ upstream to the respective start codons. In embodiments wherein the expression of one or more of the proteins is desired in a plant host, the use of all or part of the complete plant gene is desired; namely all or part of the 5′ upstream non-coding regions (promoter) together with the structural gene sequence and 3′ downstream non-coding regions may be employed. Alternatively, if a different promoter is desired, such as a promoter native to the plant host of interest or a modified promoter, i.e., having transcription initiation regions derived from one gene source and translation initiation regions derived from a different gene source, including the sequences encoding enzyme of interest, or enhanced promoters, such as double 35S CaMV promoters, the sequences may be joined together using standard techniques.

For such applications when 5′ upstream non-coding regions are obtained from other genes are desired. Tissue-specific promoters, for example may be obtained and used in accordance with the teachings of U.S. Ser. No. 07/147,781, filed Jan. 25, 1988 (now U.S. Ser. No. 07/550,804, filed Jul. 9, 1990), and U.S. Ser. No. 07/494,722 filed on or about Mar. 16, 1990 having a title “Novel Sequences Preferentially Expressed In Early Seed Development and Methods Related Thereto,” which references are hereby incorporated by reference.

Regulatory transcript termination regions may be provided in DNA constructs of this invention as well. Transcript termination regions may be provided by the DNA sequence encoding the plant enzyme or a convenient transcription termination region derived from a different gene source, for example, the transcript termination region which is naturally associated with the transcript initiation region. In particular embodiments (e.g., where the transcript termination region is from a different gene source), it will contain at least about 0.5 kb, preferably about 1-3 kb of sequence 3′ to the structural gene from which the termination region is derived.

Plant expression or transcription constructs having a plant enzye as the DNA sequence of interest for increased or decreased expression thereof may be employed with a wide variety of plant life, particularly, plant life involved in the production of vegetable oils for edible and industrial uses. Most especially preferred are temperate oilseed crops. Plants of interest include, but are not limited to, rapeseed (Canola and High Erucic Acid varieties), sunflower, safflower, cotton, Cuphea, soybean, peanut, coconut and oil palms, corn and hops. Depending on the method for introducing the recombinant constructs into the host cell, other DNA sequences may be required. Importantly, this invention is applicable to dicotyledyons and monocotyledons species alike and will be readily applicable to new and/or improved transformation and regulation techniques.

The method of transformation is not critical to the instant invention; various methods of plant transformation are currently available. As newer methods are available to transform crops, they may be directly applied hereunder. For example, many plant species naturally susceptible to Agrobacterium infection may be successfully transformed via tripartite or binary vector methods of Agrobacterium-mediated transformation. Additionally, techniques of microinjection, DNA particle bombardment, electroporation have been developed which allow for the transformation of various monocot and dicot plant species.

In developing the DNA construct, the various components of the construct or fragments thereof will normally be inserted into a convenient cloning vector which is capable of replication in a bacterial host, e.g., E. coli. Numerous vectors exist that have been described in the literature. After each cloning, the plasmid may be isolated and subjected to further manipulation, such as restriction, insertion of new fragments, ligation, deletion, insertion, resection, etc., so as to tailor the components of the desired sequence. Once the construct has been completed, it may then be transferred to an appropriate vector for further manipulation in accordance with the manner of transformation of the host cell.

Normally, included with the DNA construct will be a structural gene having the necessary regulatory regions for expression in a host and providing for selection of transformant cells. The gene may provide for resistance to a cytotoxic agent, e.g. antibiotic, heavy metal, toxin, etc., complementation providing prototrophy to an auxotrophic host, viral immunity or the like. Depending upon the number of different host species the expression construct or components thereof are introduced, one or more markers may be employed, where different conditions for selection are used for the different hosts.

It is noted that the degeneracy of the DNA code provides that some codon substitutions are permissible of DNA sequences without any corresponding modification of the amino acid sequence.

As mentioned above, the manner in which the DNA construct is introduced into the plant host is not critical to this invention. Any method which provides for efficient transformation may be employed. Various methods for plant cell transformation include the use of Ti- or Ri-plasmids, microinjection, electroporation, DNA particle bombardment, liposome fision, DNA bombardment or the like. In many instances, it will be desirable to have the construct bordered on one or both sides by T-DNA, particularly having the left and right borders, more particularly the right border. This is particularly useful when the construct uses A. tumefaciens or A. rhizogenes as a mode for transformation, although the T-DNA borders may find use with other modes of transformation.

Where Agrobacterium is used for plant cell transformation, a vector may be used which may be introduced into the Agrobacterium host for homologous recombination with T-DNA or the Ti- or Ri-plasmid present in the Agrobacterium host. The Ti- or Ri-plasmid containing the T-DNA for recombination may be armed (capable of causing gall formation) or disarmed (incapable of causing gall formation), the latter being permissible, so long as the vir genes are present in the transformed Agrobacterium host. The armed plasmid can give a mixture of normal plant cells and gall.

In some instances where Agrobacterium is used as the vehicle for transforming plant cells, the expression construct bordered by the T-DNA border(s) will be inserted into a broad host spectrum vector, there being broad host spectrum vectors described in the literature. Commonly used is pRK2 or derivatives thereof. Included with the expression construct and the T-DNA will be one or more markers, which allow for selection of transformed Agrobacterium and transformed plant cells. A number of markers have been developed for use with plant cells, such as resistance to chloramphenicol, the aminoglycoside G418, hygromycin, or the like. The particular marker employed is not essential to this invention, one or another marker being preferred depending on the particular host and the manner of construction.

For transformation of plant cells using Agrobacterium, explants may be combined and incubated with the transformed Agrobacterium for sufficient time for transformation, the bacteria killed, and the plant cells cultured in an appropriate selective medium. Once callus forms, shoot formation can be encouraged by employing the appropriate plant hormones in accordance with known methods and the shoots transferred to rooting medium for regeneration of plants. The plants may then be grown to seed and the seed used to establish repetitive generations and for isolation of vegetable oils.

Once a transgenic plant is obtained which is capable of producing seed having a modified fatty acid composition and/or accumulation level, traditional plant breeding techniques, including methods of mutagensis, may be employed to further manipulate the fatty acid composition. Alternatively, additional foreign fatty acid modifying DNA sequence may be introduced via genetic engineering to further manipulate the fatty acid composition. It is noted that the method of transformation is not critical to this invention. However, the use of genetic engineering plant transformation methods (e.g., to insert a single desired DNA sequence) is critical. Heretofore, the ability to modify the growth characteristics of plants was limited to the introduction of traits that could be sexually transferred during plant crosses or viable traits generated through mutagensis. Through the use of genetic engineering techniques which permit the introduction of inter-species genetic information and the means to regulate the tissue-specific expression of endogenous genes, a new method is available for the growth engineering of plants using the inventive nucleic acids and proteins. There is the potential for the development of novel plant upon application of the tools described herein.

One may choose to provide for the transcription or transcription and translation of one or more other sequences of interest in concert with the expression of a plant enzyme in a plant host cell. In particular, the reduced expression of one or more brassinosteroid biosyntetic enzymes may be preferred in some applications.

For providing a plant transformed for the combined effect of more than one nucleic acid sequence of interest, typically, but not necessarily a separate nucleic acid construct will be provided for each. The constructs, as described above, contain transcriptional or transcriptional or transcriptional and translational regulatory control regions. One skilled in the art will be able to determine regulatory sequences to provide for a desired timing and tissue specificity appropriate to the final product in accord with the above principles (e.g., respective expression or anti-sense constructs). When two or more constructs are to be employed, whether they are both related to the same brassinosteroid biosyntetic enzyme sequence or a different brassinosteroid biosyntetic enzyme sequence, it may be desired that different regulatory sequences be employed in each cassette to reduce spontaneous homologous recombination between sequences. The constructs may be introduced into the host cells by the same or different methods, including the introduction of such a trait by crossing transgenic plants via traditional plant breeding methods, so long as the resulting product is a plant having both characteristics integrated into its genome.

Preferably, an inventive plant enzyme includes any sequence of amino acids, such as a protein, polypeptide, or peptide fragment, obtainable from a plant source which is capable of catalyzing the respective biological activity in a plant host cell, i.e., in vivo, or in a plant cell-like environment, i.e., in vitro. “A plant cell-like environment” means that any necessary conditions are available in an environment (i.e., such factors as temperatures, pH, lack of inhibiting substances) which will permit the enzyme to function.

Biologically Active Variants

Variants of the altered plant brassinosteroid biosyntetic enzymes disclosed herein have substantial utility in various inventive aspects. Variants can be naturally or non-naturally occurring. Naturally occurring variants are found in plants or other species and comprise amino acid sequences which are substantially identical to the amino acid sequences herein, and include natural sequence polymorphisms. Species homologs of the protein can be obtained using subgenomic polynucleotides of the invention, as described herein, to make suitable probes or primers for screening cDNA expression libraries from other plant species, or organisms, identifying cDNAs which encode homologs of the protein, and expressing the cDNAs as is known in the art.

Non-naturally occurring variants which retain substantially the same biological activities as altered naturally occurring protein variants, are also included here. Preferably, naturally or non-naturally occurring variants have amino acid sequences which are at least 75%, at least 85%, at least 90%, or at least 95% identical to the amino acid sequences shown herein. More preferably, the molecules are at least 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. 2:482-489, 1981.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R.. §§.1.821-1.822, abbreviations for amino acid residues are shown in Table A:

TABLE A
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Praline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

It should be noted that all amino acid residue sequences represented herein by a formula have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R.. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

Guidance in determining which amino acid residues can be substituted, inserted, or deleted without abolishing biological or immunological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

Such substitutions may be made in accordance with those set forth in TABLE B as follows:

TABLE B

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative (or non-conservative) substitutions.

Variants of the brassinosteroid biosyntetic enzymes disclosed herein include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

A subset of mutants, called muteins, is a group of polypeptides in which neutral amino acids, such as serines, are substituted for cysteine residues which do not participate in disulfide bonds. These mutants may be stable over a broader temperature range than native secreted proteins (Mark et al., U.S. Pat. No. 4,959,314).

In particular aspects, amino acid changes in the brassinosteroid biosyntetic enzyme variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of Herstatin and/or RBD Int8 polypeptide protein or polypeptide variants are of the same type as a protein comprising the amino acid sequence encoded by the nucleotide sequences shown in SEQ ID NO:1 or 2, although the properties and functions of variants can differ in degree.

Brassinosteroid biosyntetic enzyme polypeptide variants include glycosylated forms, aggregative conjugates with other molecules, and covalent conjugates with unrelated chemical moieties (e.g., pegylated molecules). Truncations or deletions of regions which do not preclude functional activity of the proteins are also variants. Covalent variants can be prepared by linking functionalities to groups which are found in the amino acid chain or at the N- or C-terminal residue, as is known in the art.

It will be recognized in the art that some amino acid sequence of the brassinosteroid biosyntetic enzymes of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors (Ostade et al., Nature 361:266-268, 1993). Thus, brassinosteroid biosyntetic enzyme polypeptides of the present inventive aspects may include one or more amino acid substitutions, deletions or additions, either from natural mutations or human manipulation.

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing formulations.

Amino acids in the inventive brassinosteroid biosyntetic enzyme polypeptides that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904, 1992 and de Vos et al. Science 255:306-312,1992).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

In addition, pegylation brassinosteroid biosyntetic enzyme polypeptides and/or muteins is expected to provide such improved properties as increased half-life, solubility, and protease resistance. Pegylation is well known in the art.

Functional brassinosteroid biosyntetic enzyme polypeptides, and functional variants thereof, are those proteins that display one or more of the biological activities of their resepective brassinosteroid biosyntetic enzymes. In particular aspects, such functional variants and portions thereof have at least 85%, at least 90%, at least 95%, at least 97%, at least 98%, or at least 99% homology to the respective brassinosteroid biosyntetic enzyme or respective portions thereof.

Fusion Proteins

Fusion proteins comprising proteins or polypeptide fragments of brassinosteroid biosyntetic enzymes can also be constructed. Fusion proteins are useful for generating antibodies against amino acid sequences and for use in various targeting and assay systems. For example, fusion proteins can be used to identify proteins which interact with inventive brassinosteroid biosyntetic enzyme or which interfere with the respective biological functions. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence can be used.

A fusion protein comprises two protein segments fused together by means of a peptide bond. Amino acid sequences for use in fusion proteins of the invention can be utilize the inventive brassinosteroid biosyntetic enzyme or can be prepared from biologically active or inactive variants of inventive brassinosteroid biosyntetic enzyme, such as those described herein. The first protein segment can include a full-length inventive brassinosteroid biosyntetic enzyme. Other first protein segments can consist of contiguous sub-amino acid regions.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and viral protein fusions. Other fusions are possible, as would be recognized by one skilled in the art.

These fusions can be made, for example, by covalently linking two protein segments or by standard procedures in the art of molecular biology. Recombinant DNA methods can be used to prepare fusion proteins, for example, by making a DNA construct which comprises a coding region for the protein sequence of one of the brassinosteroid biosyntetic enzymes in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Particular aspects provide a novel isolated DNA sequences encoding an altered protein or polypeptide comprising or consisting of an amino acid sequence as disclosed herein, respective biologically active portions thereof, and respective sequences having at least 85%, at least 90%, at least 95%, at at least 97%, at least 98%, or at least 99% homology thereto. In certain aspects, the isolated coding sequence is selected from the group consisting of Arabidopsis thaliana sequences as disclosed herein, and sequences having at least 85%, at least 90%, at least 95%, at at least 97%, at least 98%, or at least 99% homology thereto.

Additional aspects provide a novel isolated protein or polypeptide, comprising or consisting of an amino acid sequence selected from the group consisting of consisting of Arabidopsis thaliana sequences as disclosed herein, respective biologically active portions thereof, and respective sequences having at least 85%, at least 90%, at least 95%, at at least 97%, at least 98%, or at least 99% homology thereto. Certain embodiments provide for fusion proteins comprising these novel sequences.

Further aspects provide a transfected cell, comprising at least one expression vector having a DNA sequence that encodes upon expression a protein or polypeptide comprising or consisting of an amino acid sequence selected from the group consisting Arabidopsis thaliana sequences as disclosed herein, respective biologically active portions thereof, and respective sequences having at least 85%, at least 90%, at least 95%, at at least 97%, at least 98%, or at least 99% homology thereto, and respective fusion proteins thereof operatively associated with a heterologous polypeptide. Preferably, the DNA sequence comprises a sequence selected from the group consisting of Arabidopsis thaliana sequences as disclosed herein, respective biologically active portions thereof, and respective sequences having at least 85%, at least 90%, at least 95%, at at least 97%, at least 98%, or at least 99% homology thereto.

Additional embodiments provide a novel antibody or epitope-binding fragment thereof specific for an altered amino acid sequence as disclosed herein, respective epitope-bearing portions thereof, and respective sequences having at least 85%, at least 90%, at least 95%, at at least 97%, at least 98%, or at least 99% homology thereto. Preferably, the antibody or epitope-binding fragment thereof is a monoclonal antibody or antigen-binding fragment thereof.

Arabidopsis thaliana. Arabidopsis thaliana is an ideal plant model for studying plant biosynthesis (e.g., oil/seed oil, hormones, etc.) because of its short life cycle, ease of growth, large seed yield, and its art-recognized similarity to other crop species (e.g., oil crop species), providing a validated model system that is applicable to other crop species. Arabidopsis is easily transformed with foreign DNA via Agrobacterium tumefaciens infection. This process, as described herein can be used for manipulating the Arabidopsis Bto mimic the castor bean ricinoleic production pathway.

Exemplary Preferred Embodiments:

Particular aspect provide a method for modulating plant characteristics, comprising expressing within a plant of at least one brassinosteroid biosyntetic enzyme comprising at least one alteration of the amino acid sequence thereof, wherein the at least one amino acid sequence alteration modifies a calmodulin-binding activity of the at least one enzyme, wherein at least one plant characteristic is modified. In certain embodiments, the at least one amino acid sequence alteration comprises an amino acid substitution, deletion or insertion. In particular aspects, the at least one amino acid sequence alteration comprises an amino acid substitution. In certain aspects, the at least one amino acid sequence alteration comprises a non-conservative amino acid substitution. In other aspects, the sequence alteration is conservative. In certain embodiments, the at least one alteration in amino acid sequence is in a calmodulin-binding domain of the at least one calmodulin-binding brassinosteroid biosyntetic enzyme.

In particular aspects, the at least one plant characteristic comprises at least one of size, growth, productivity, and fertility. In certain embodiments, the at least one plant characteristic comprises dwarfism or semi-dwarfism. Preferably, the characteristic is dwarfism or semi-dwarfism and is associated with another characteristics such as enhance productivity of plant (e.g, more seeds, flowers, fruit, oil, etc).

In particular embodiments, the at least one brassinosteroid biosyntetic enzyme comprises at least one selected from the group consisting of DWF1, DWF4, DWF5, CPD and plant orthologs thereof. In certain aspects, the at least one brassinosteroid biosyntetic enzyme comprises at least one selected from the group consisting of DWFV1, and calmodulin-binding orthologs thereof. Preferably, the orthologs are plant orthologs.

In certain aspects, expressing comprises expressing within a plant of a plurality brassinosteroid biosyntetic enzymes, in each case comprising at least one alteration of the amino acid sequence thereof. In particular embodiments, expressing within a plant of at least one brassinosteroid biosyntetic enzyme comprising at least one alteration of the amino acid sequence thereof comprises said expressing in the presence of expression of a respective endogenous non-altered calmodulin-binding brassinosteroid biosyntetic enzyme. In certain aspects, expressing within a plant of at least one brassinosteroid biosyntetic enzyme comprising at least one alteration of the amino acid sequence thereof comprises said expressing in the absence of, or in the presence of reduced expression of a respective endogenous non-altered calmodulin-binding brassinosteroid biosyntetic enzyme. In particular embodiments, expressing within a plant of at least one brassinosteroid biosyntetic enzyme comprising at least one alteration of the amino acid sequence thereof comprises said expressing in the presence of expression of a mutant or inactivated respective endogenous brassinosteroid biosyntetic enzyme that may or may not have calmodulin-binding acitity.

Preferably, the plant is a crop plant. In particular aspects, the crop plant is Hops. In particular aspects, the crop plant is Hops and the characteristic is dwarfism or semi-dwarfism and the dwarf or semi-dwarf Hops are more efficient at producing ‘cones’ relative to non-modified counterparts.

Additional aspects provide a life form (e.g., plant) generated by the method of claim 1. Preferably, the plant is a crop plant. In particular aspects, the crop plant is Hops. In particular aspects, the crop plant is Hops and the characteristic is dwarfism or semi-dwarfism and the dwarf or semi-dwarf Hops are more efficient at producing ‘cones’ relative to non-modified counterparts.

Yet further embodiments provide a method for modulating the activity of a brassinosteroid biosyntetic enzyme, comprising introduction into the amino acid sequence of a calmodulin-binding brassinosteroid biosyntetic enzyme at least one amino acid sequence alteration, wherein the at least one amino acid sequence alteration modifies a calmodulin-binding activity of the at least one enzyme. In particular aspects, the at least one amino acid sequence alteration comprises an amino acid substitution, deletion or insertion. Preferably, the at least one amino acid sequence alteration comprises an amino acid substitution. Preferably, at least one amino acid sequence alteration comprises a non-conservative amino acid substitution. In particular aspects, the at least one alteration in amino acid sequence is in a calmodulin-binding domain of the at least one calmodulin-binding brassinosteroid biosyntetic enzyme. In particular aspects, the calmodulin-binding brassinosteroid biosyntetic enzyme is selected from the group consisting of DWF1, DWF4, DWF5, CPD and calmodulin-binding orthologs thereof. Preferably, the ortholog is a plant ortholog. In particular embodiments, the at least one calmodulin-binding brassinosteroid biosyntetic enzyme comprises at least one selected from the group consisting of DWF1, and calmodulin-binding orthologs thereof. Preferably, the ortholog is a plant ortholog.

Further aspects provide a nucleic acid or expression vector encoding at least one brassinosteroid biosyntetic enzyme comprising at least one alteration of the amino acid sequence thereof, wherein the at least one amino acid sequence alteration modifies a calmodulin-binding activity of the at least one enzyme.

Additional aspects provide a cell transfected with a nucleic acid or expression vector encoding at least one brassinosteroid biosyntetic enzyme comprising at least one alteration of the amino acid sequence thereof, wherein the at least one amino acid sequence alteration modifies a calmodulin-binding activity of the at least one enzyme. Preferably, the cell is a plant cell.

Yet further aspects, provide an isolated protein, comprising at least one alteration of the amino acid sequence of a brassinosteroid biosyntetic enzyme, wherein the at least one amino acid sequence alteration modifies a calmodulin-binding activity of the at least one enzyme. In particular aspects, the brassinosteroid biosyntetic enzyme is selected from the group consisting of DWF1, DWF4, DWF5, CPD and calmodulin-binding orthologs thereof. Preferably, the ortholog is a plant ortholog. In particular embodiments, the at least one brassinosteroid biosyntetic enzyme comprises at least one selected from the group consisting of DWF1, and calmodulin-binding orthologs thereof. Preferably, the ortholog is a plant ortholog. Yet additional aspects provide a method for modulating the expression of at least one endogenous brassinosteroid biosyntetic enzyme, comprising expressing a recombinant enzyme as disclosed herein.

### EXAMPLE 1

### DWF1 was shown herein to be a Calmodulin (CaM) Binding Protein, and the CaM-Binding Domain thereof was Identified and Characterized

An Arabidopsis complementary DNA expression library was screened using ³⁵S-calmodulin (CaM) as a probe, and a positive clone was isolated that included the 44-amino-acid carboxy terminus of Arabidopsis DWF1 (At3g19820).

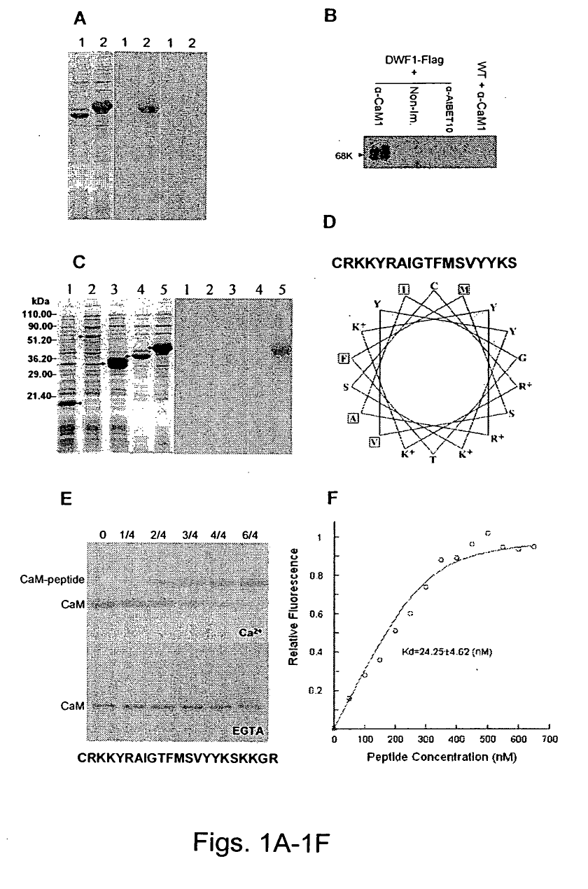

To confirm further the CaM-binding ability of DWF1, the positive clone from the library screening was expressed in Escherichia coli and tested for ³⁵S-CaM binding¹⁵ in the presence of Ca²⁺, EGTA, a Ca²⁺ chelator, or Mg²⁺. Recombinant DWF1 bound to ³⁵S-CaM in the presence of Ca²⁺, but not EGTA (FIG. 1a) or Mg²⁺ (data not shown), indicating that DWF1 binds to CaM in a Ca²⁺-dependent manner. C-terminal Flag-tagged DWF1 overexpression lines of Arabidopsis were produced to study the DWF1-CAM interaction in vivo. Because of the dynamic nature of the interaction between DWF1 and Ca²⁺/CaM, co-immunoprecipitation using a prior in vivo crosslinking strategy¹⁶ was adapted to verify their interaction under physiological conditions.

Co-immunoprecipitation with prior crosslinking. One gram of rosette leaves from 3-4-week-old Arabidopsis plants expressing DWF1-Flag were harvested, washed briefly, cut into ˜5-mm² pieces, soaked in 40 ml ice-cold lysis buffer (10 mM HEPES-KOH, pH 7.4, 12.5% sucrose, 10 mM KAC, 3 mM MgCl₂, 1 mM CaCl₂) supplemented with 0.01% detergent Silwet-77 and 1% formaldehyde, and subjected to vacuum and release for two cycles (5 min each). After incubating at 4° C. for 30 min, the leaves were washed two times for 20 min with 50 ml lysis buffer supplemented with 0.3 M glycine at 4° C., and stored at −80° C. until further use. The fixed leaves were ground in liquid N₂ to a fine powder, re-suspended in four volumes of lysis buffer supplemented with NaN₃ and 1×Roche complete protease inhibitors, and centrifuiged two times at 4° C., 12,000 g for 10 min to obtain a clear total protein extract. Polyclonal anti-CaM1 antibody (2 μg, Santa Cruz) was added to 500 μg total protein and the mixture was shaken at room temperature for 1 h. Pre-blocked protein-G plus agarose was added and shaken for another hour. The immunocomplexes was washed three times in lysis buffer supplemented with 1×Roche complete protease inhibitors and 150 mM NaCl, re-suspended in 30 μl 2×SDS loading buffer, boiled for 25 min to reverse the crosslinking induced by formaldehyde, separated on SDS-PAGE, and immuno-detected with anti-Flag M2 monoclonal antibody. Total protein from wild-type plants, non-immune serum and anti-AtBET10 (ref. 15) antibody were used as negative controls.

The results revealed that Flag-tagged DWF1 interacted with CaM in vivo (FIG. 1b).

To map its CaM-binding domain (CaMBD), three different segments that covered the entire length of the DWF1 gene were expressed and tested for ³⁵S-CaM binding, and the results indicated that there is only one CaMBD in the 44-amino-acid C terminus (FIG. 1c). The CaMBD is typically comprised of a stretch of 12-30 contiguous amino acids that contain a positive net charge and have a propensity to form an amphiphilic α-helix⁴. Helical wheel projection indicated that the amino acid region 518-539 is the CaMBD (FIG. 1d). A peptide corresponding to 518-539 was synthesized, and its CaM-binding ability was confirmed using PCM6 (ref. 17) in a gel mobility shift assay¹⁵ (FIG. 1e). It was further confirmed to bind to conserved CaM isoforms such as PCM1 (ref. 17) and bovine CaM, but not to Arabidopsis CaM9, a divergent CaM isoform (ref. 18 and data not shown).

To estimate the affinity of the interaction between DWF1 and Ca²⁺/CaM, fluorescence titration of dansyl-CaM (Sigma) in the presence of increasing amounts of the DWF1 CaM-binding peptide (518-539) was performed as described¹⁹, and the dissociation constant (K_{d}) was calculated to be 24.25±4.62 nM (FIG. 1f).

CaM is a well-characterized Ca²⁺ sensor protein and it can bind to and regulate the function of various CaM-binding proteins by changing their conformation^{4,5}. Overexpression of other brassinosteroid biosynthetic genes such as DWF5 (ref. 20), DET2 (ref. 21), DWF4 (ref. 10) and CPD²² resulted in a hypermorphic phenotype with increased vegetative growth. Surprisingly, overexpression of DWF1 did not show any phenotypic change⁶, implying that an unknown condition was not met for the overexpressed DWF1 to produce a hypermorphic phenotype. Applicants have previously found that upregulated expression of one CaM isoform (PCM1) resulted in increased growth and apical dominance in potato¹⁷, similar to the results of brassinosteroid biosynthetic gene overexpression. The identification herein of DWF1 as a CaM-binding protein indicates the presence of a regulatory mechanism exerted on DWF1 at the protein level.

### EXAMPLE 2

### According to Additional Aspects, Loss-Of-Function (LOF) Mutations in the Calmodulin-Binding Domaine (CaMBD) led to the Functional Loss of the Entire Protein in in Planta Complementation Tests)

Because there are no established enzymatic procedures to test the function of DWF1, mutation and complementation strategies were used to study the effect of CaM-binding on DWF1 function.

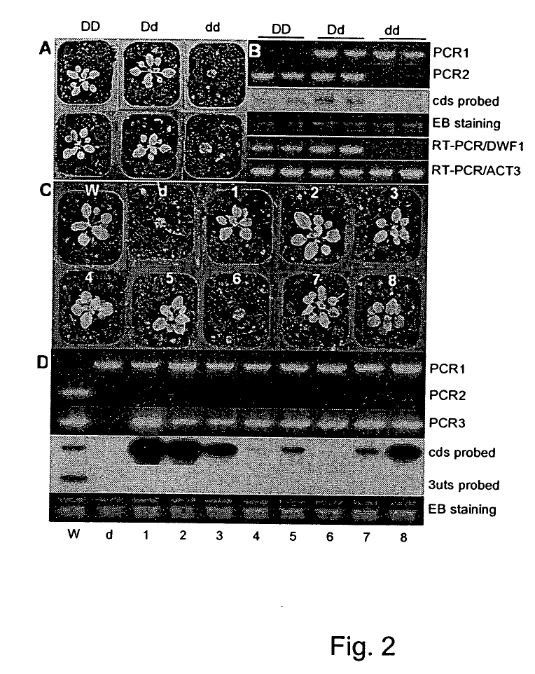

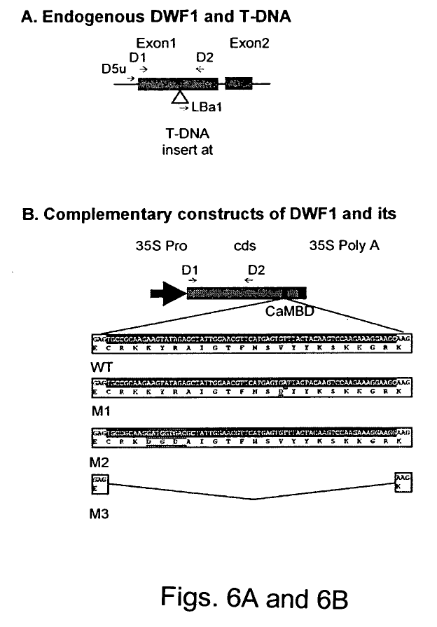

To circumvent the infertility problem associated with homozygous dwf1 mutants^{6,7}, a heterozygous dwf1 line (SALK_{—}006932, ref. 23) was chosen for transformation. A T-DNA insert is shown in FIG. 6, panel A, and detailed characterizations confirmed that it is a heterozygous dwf1 null mutation line (FIG. 2a, b).

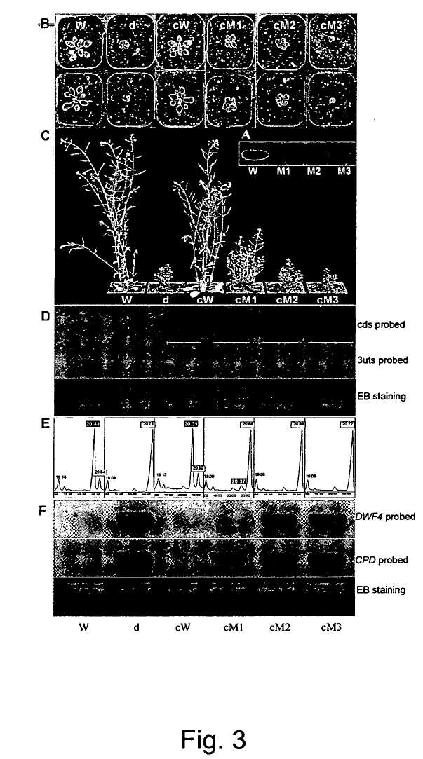

Specifically, heterozygous SALK_{—}006932 plants were transformed with a 35S::DWF1 construct (FIG. 6, panel B). Almost all of the T1 plants showed a normal phenotype. Using polymerase chain reaction (PCR) analysis, one dwarf and seven normal plants were identified that carried at least one copy of the 35S::DWF1 complementary construct and were homozygous knockout at the endogenous DWF1 loci. The dwarfism in the dwarf plant was confirmed to be caused by a failure in expression of the transgene (FIG. 2c, d). The overexpressed (numbers 1, 2, 3, 8) and equally expressed (numbers 5, 7) lines grew like the wild-type, confirming a previous report⁶. The underexpressed line (number 4) exhibited an appearance very similar to the wild-type with a slightly shorter petiole and rounder leaves, whereas the silenced line (number 6) showed a complete dwarf phenotype (FIG. 2c), indicating that the phenotype of the DWF1 complemented plants (cW) correlated with the transgene expression level in the underexpressed conditions. At the adult stage, 7 out of the 8 complemented lines reached similar levels of height and fertility as that of wild-type plants (FIG. 3c).

These results demonstrated that the 35S promoter-driven DWF1 gene can completely rescue the dwarf phenotype of the dwf1 null mutation.

Mutants. Three mutants were produced to disrupt the conserved secondary structure of the CaMBD of DWF1 (FIG. 6, Panel B): V531D (M1), aimed at disturbing the hydrophobic surface; KYR521-523DGD (M2, K521, Y522 and R523 are changed to D521, G522 and D523), aimed at changing the net surface charges; and Δ518-539 (M3), a total deletion of the CaMBD. The effects of these mutations were tested, and the results revealed that the CaM-binding ability was markedly decreased for V531D and was undetectable for KYR52-5231DGD and Δ518-539 (FIG. 3a). A similar strategy was used to deliver the mutant dwf1 complimentary constructs (FIG. 6, Panel B) into the heterozygous dwf1 plants. Visual checks identified 18, 11 and 19 dwarf plants in the T1 population carrying 35S::DWF1(V531D), 35S::DWF1(KYR521-523DGD) and 35S::DWF1(Δ518-539) complimentary constructs, respectively. These plants were confirmed to carry at least one copy of the transgene and the homozygous knockout at the endogenous DWF1 loci.

To ensure a reliable comparison of the complementary effects of normal DWF1 and the three mutants, T2 plants with similar expression levels were compared (FIG. 3b-d). In contrast, typical DWF1(V531D) complemented dwarf mutants (cM1) showed some degree of phenotype rescue, with round, expanded leaves but unextended petioles at the rosette stage and bushy flowering stems reaching up to 40% of the height of the wild-type plant. Seeds were produced in most of the slightly shorter siliques of cM1 plants (FIG. 3b, c). Typical DWF1(KYR521-523DGD) complemented plants (cM2) showed very slight phenotypic rescue, with expanded, round and curved leaves and unextended petioles at the rosette stage, as well as bushy and slightly longer flower stems, and seeds in most of the siliques (FIG. 3b, c). All of the DWF1(Δ518-539) complemented dwf1 mutants (cM3) grew like the uncomplemented dwf1 mutants (FIG. 3b, c). The dwarf phenotypes of cM1, cM2, cM3 and the dwf1 mutant were rescued by exogenous application of brassinolide (data not shown).

These results demonstrate that loss-of-function mutations in the CaMBD of DWF1 leads to the functional loss of the whole protein in complementation tests.

DWF1 catalyses the conversion of isofucosterol to sitosterol and 24-methylenecholesterol to campesterol^{6,7}. The endogenous levels of sitosterol/isofiicosterol or campesterol/24-methylenecholesterol in wild-type, dwarf and complemented plants were analysed using gas chromatography-mass spectrometry (GC-MS) to monitor the function of the complementing DWF1, DWF1(V531D), DWF1(KYR521-523DGD) and DWF1(Δ518-539) carried in cW, cM1, cM2 and cM3 plants, respectively.

Sterol analysis. Six- to seven-week-old greenhouse-grown plants were harvested and ground into a fine powder in liquid nitrogen. Aliquots of 100 mg ground material were sponified in 1 N NaOH, 70% ethanol at 70° C. for 2 h. The sterols were extracted three times with 500 μl hexane, and the combined extracts were dried and dissolved in 100 μl of methylene chloride. GC-MS analyses were conducted on an Agilent 6890 GC system with 5973N mass selective detector. A 1 μl aliquot of methylene chloride extract containing the underivatized sterols was loaded by cool on column injection on a Zebron (Phenomenex) ZB-5 column, 30 m×0.25 mm internal diameter with 0.25 μm film thickness. Chromatography was accomplished using He as the carrier gas at a flow rate of 0.7 ml min⁻¹. The column oven temperature was programmed to run from 40° C. to 300° C. increased at 20° C. min⁻¹, with a hold for 10 min at 300° C. Identification and quantification of sterols was accomplished by comparison to authentic standards based on the retention time, peak area and mass fragmentation pattern.

The results (FIG. 3e) indicate that the function of the DWF1 enzyme in cW plants was recovered to a level comparable to that in wild-type plants. In cM1 plants DWF1 function was restored to some extent, but the recovery of DWF1 function in cM2 and cM3 plants was almost non-existent. To confirm further that the endogenous brassinosteroid levels are also correlated with complementation events, applicants monitored the expression of brassinosteroid marker genes such as CPD and DWF4, which are known to be feedback controlled by brassinosteroid signals^{8,9}. The results revealed that in wild-type and cW plants the expression of both DWF4 and CPD was very low, indicating a normal brassinosteroid level; however, in dwf1 mutant and cM1, cM2 and cM3 plants expression of both DWF4 and CPD was upregulated, indicating decreased levels of endogenous brassinosteroids (FIG. 3f). Furthermore, transcription of DWF4 and CPD correlated with the phenotypes of the corresponding plants and their endogenous sitosterol/isofucosterol levels, as revealed by GC-MS analysis.

### EXAMPLE 3

### According to further Inventive Aspects, DWF4, DWF5, and CPD were also found to be Ca²⁺/CaM-Binding Proteins

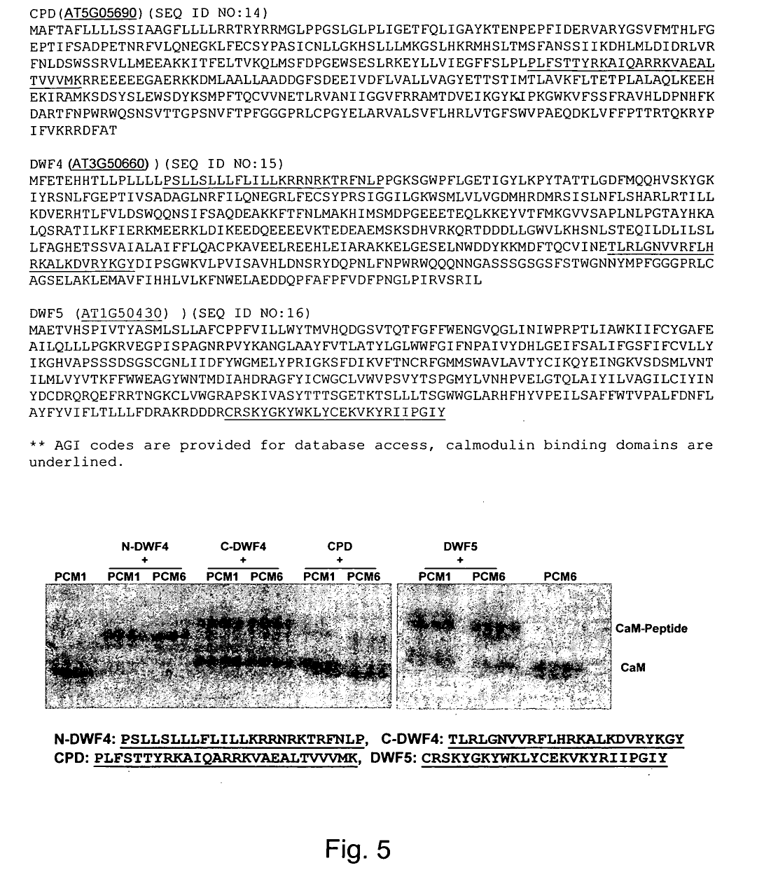

DWF1Ca²⁺ is an important second messenger and an integral part of the cellular signalling system in plants^{5,29}. Applicants were thus prompted to survey the CaM-binding properties of other proteins involved in brassinosteroid biosynthesis. Interestingly, three important enzymes, DWF4 (AT3G50660) (SEQ ID NO:15), DWF5 (AT1G50430) (SEQ ID NO:16), and CPD (AT5G05690) (SEQ ID NO:14), were also found to be Ca²⁺/CaM-binding proteins (FIG. 5), especially DWF4, which has two CaM-binding domains with markedly different affinities for Ca²⁺/CaM (unpublished data).

Therefore, the results described herein indicate not only that Ca²⁺/CaM binding is critical for DWARF1 function in brassinosteroid biosynthesis and plant growth, but that Ca²⁺/CaM has complex and wide-ranging regulatory controls on the biosynthesis of brassinosteroid, and that DWF1, DWF4, DWF5, and CPD are examples of these regulatory steps.

### EXAMPLE 4

### According to Additional Aspects, the DWF1 CaMBD was found to be Conserved in the Orthologous Genes of a broad array of Plants, Indicating the Applicability of the Presently Disclosed Compositions and Methods to a Broad array of Plants, including Crop Plants)

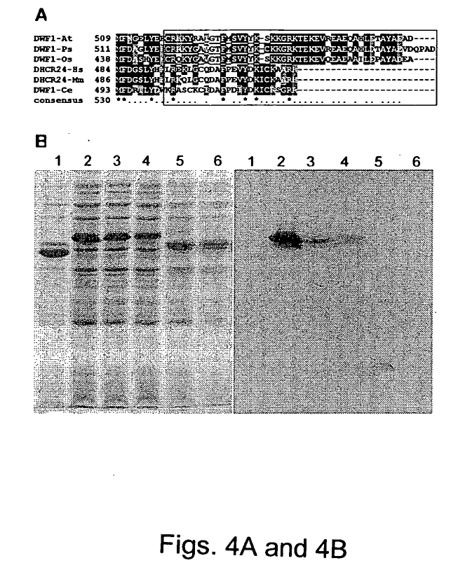

DWF1 orthologues, as well as those of DWF4, DWF5, and CPD, exist in both plants and animals. Protein sequences of DWF1 and its orthologues from pea, rice, human, mouse and Caenorhabditis elegans were aligned and the C-terminal CaMBD was found to be conserved in plants but not animals (FIG. 4a). The boxed parts in FIG. 4a—the comparable parts of DWF1 and its orthologues from Arabidopsis, pea, rice, human and mouse—were expressed in E. coli, and the ³⁵S-CaM-binding assay demonstrated that only the C terminus of plant DWF1 orthologues bind to Ca²⁺/CaM, indicating that the orthologues of DWF1 in plants are broadly regulated by Ca²⁺/CaM through the C-terminal region. Likewise, the data indicate that the orthologues of DWF4, DWF5, and CPD in plants are broadly regulated by Ca²⁺/CaM through the respective calmodulin-binding domains.

Additionally, characterization of brassinosteroid synthetic mutants such as det2 (ref. 24) and cpd²² linked the light signal to brassinosteroids, and this connection has been established by genetic and biochemical analyses of the brassinosteroid signalling processes^{2,3}. Light has been shown to induce cytosolic Ca²⁺ changes and subsequent physiological responses related to photomorphogensis, and probably phototropism^{25,26}, indicating that light signal can be transduced through Ca²⁺. The Arabidopsis det3 mutant expresses remarkably reduced subunit C of the vacuolar H⁺-ATPase (VHA-C) due to a mutation in this gene, and its de-etiolated phenotype and dwarfism indicate a defect in hormone signalling²⁷. The det3 mutant was later found to have altered Ca²⁺ oscillation and guard cell response to some of the tested signals, probably caused by the deficient VHA-C activity²⁸.

The present results therefore also indicate that a defective growth phenotype and possibly altered hormone signalling in det3 is connected to altered Ca²⁺ responses.

### EXAMPLE 5

### According to Additional Aspects Semi-Dwarf Hop Plants are Provided

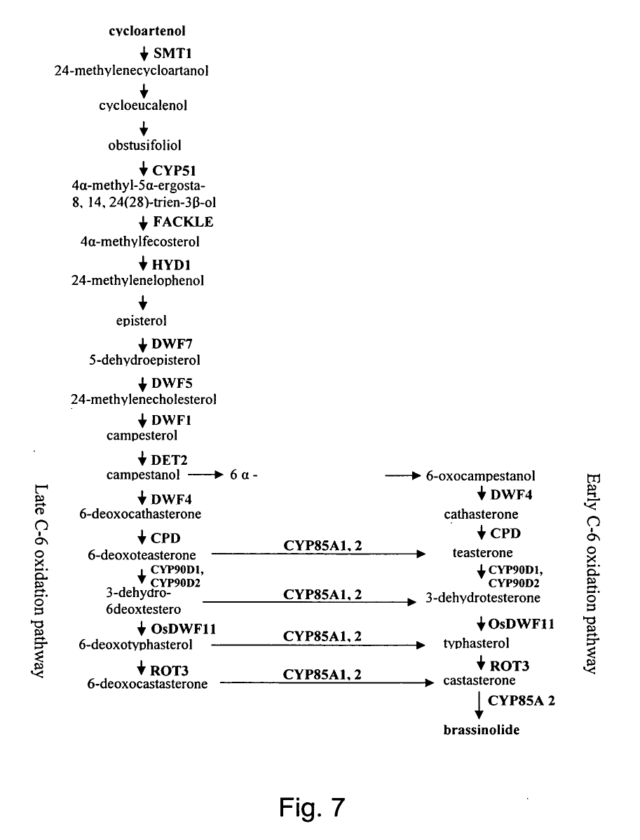

Brassinosteroids (BRs) are a class of plant hormones that play major roles throughout the plant kingdom. The BR biosynthetic pathway (FIG. 7), which ends in the production of brassinolide, was found to be highly conserved in both dicots and monocotyledons (Fujioka and Yokota, 2003).

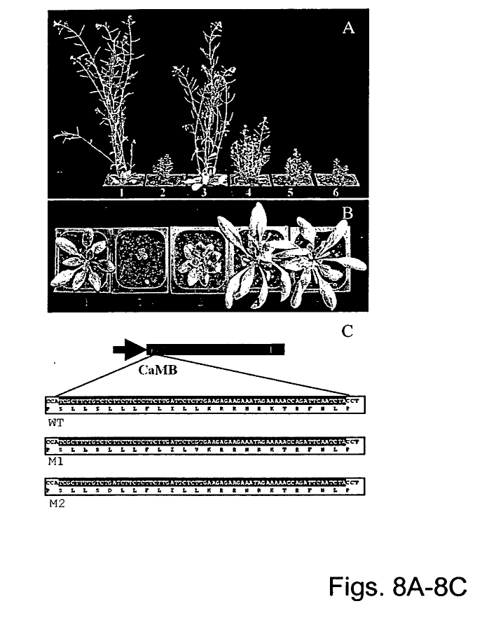

As described herein above, applicants have demonstrated that DWF1, an enzyme which converts 24-methylenechesterol into campesterol (FIG. 7) is a calmodulin-binding protein and the activity of DWF1 is regulate by calcium/calmodulin. Likewise DWF4, DWF5, and CPD) were demonstrated to be calmodulin-binding proteins. For example, molecular genetic analysis using site-directed and deletion mutants revealed that loss of calmodulin-binding totally abolished the function of DWFV1 in planta, while partial loss of calmodulin-binding resulted in a partial dwarf phenotype in complementation studies (FIG. 8A). The disclosed results demonstrate that BR synthesis is essential for normal plant growth and development, and that plant stature is directly correlated with the endogenous BR level and the activities of BR biosynthetic proteins in plants.

Hop (Humulus lupulus L.) is a perennial, climbing plant, and it is a dioecious plant. The female inflorescence (cones) are an important ingredient in the production of beer. According to additional aspects, a dwarf or semi-dwarf plant that produces a higher proportion of inflorescence is provided. For example, dwarf or semi-dwarf Hop plants are provided, wherein the at least one of the respective calmodulin-binding acitivities of the corresponding Hop orthologs to DWF1, DWF4, DWF5, and CPD are modulated according to the presently disclosed methods wherein at least one of the respective CaMBDs is alterted to modulate the calmodulin-binding acitivities of at least one of the respective Hop orthologs to DWF1, DWF4, DWF5, and CPD.

Indeed applicants results show (FIG. 8B) that semi-dwarf plants can be generated using more than one approach. For example, partial dwarf Arabidopsis plants were generated through manipulation of the DWF4 CaMBD regulatory domain. FIG. 8B, pot 3, shows a semi-dwarf generated by complementing a DWF4 null mutant with a DWF4 mutant having a L to D change at amino acid 21 of DWF4, where this amino acid position lies within the N-termainl CaMBD of DWF4. By contrast, overexpression of a mutant having a L to V change at amino acid 28 (also within the N-termainl CaMBD of DWF4) showed enhanced growth.

Therefore, according to additional aspects and based on the conserved BR biosynthesis among higher plants, dwarf or semi-dwarf Hop plants, or Hop plants with altered growth or productivity characteristics are provided by modulating the activity of the CaMBD of the hop homologs of dwf5, dwf1, det2, dwf4 or cpd (e.g., to produce hops with varying degrees of dwarfism).

For example, Hop orthologs/homologs of dwf5, dwf1, det2, dwf4 or cpd gene are cloned, constructs comprising mutant CaMBDs are generated by standard methods, and inserted into hop plants to modulate the expression and/or activity of BR biosynthetic genes. The techniques necessary for such manipulation are known in the art, and, for example production of transgenic Hop using A. tumefaciens-mediated transformation has been published (Horlemann et al., 2003). According to particular aspects, using these methods, Hops with a decreased stature and/or productivity are provided. Additionally, combinations of dwf5, dwf1, det2, dwf4 or cpd mutant proteins are encompassed within the present conception, as well as constructing and then complementing particular HOP BR enzyme null mutants with one or more respective CaMBD mutants of dwf5, dwf1, det2, dwf4 or cpd, whereby the degree of dwarfism or productivity can be customized or fine tuned to achieve desirable properties (e.g., regain fertility, or pest or disease resistance, etc.) not attainable by current traditional dwarf breeding methods. Furthermore, enzyme mutants according to present invention can be obtained by reverse genetic strategies such as TILLING for plant functional genomics (see., e.g., McCallum et al., Plant Physiology, 123:439-442, 2000; and Henikoffet al., Plant Pysiology, 135:630-636, 2004, both of which are incorporated by reference in their entirety.

SEQUENCE LISTING CROSS-REFERENCE

| SEQ | | Sequence | Accession |
|---|---|---|---|
| ID NO | Name of Sequence | Type | No. |
| 1 | Homo sapiens 24-dehydro- | DNA | NM_014762 |
| | cholesterol reductase | | |
| | (DHCR24), mRNA | | |
| 2 | 24-dehydrocholesterol | AA | NP_055577.1 |
| | reductase precursor (Homo | | |
| | sapiens) | | |
| 3 | Zea mays brassinosteroid | DNA | AY523572 |
| | biosynthesis-like protein | | |
| | (DWF1) mRNA, complete cds | | |
| 4 | brassinosteroid biosynthesis- | AA | AAS90832 |
| | like protein (Zea mays) | | |
| 5 | Arabidopsis thaliana DWF1 | DNA | NM_180285 |
| | (DIMINUTO 1); catalytic | | |
| | (DWF1) mRNA, complete cds | | |
| 6 | DWF1 (DIMINUTO 1); cata- | AA | NP_850616 |
| | lytic [Arabidopsis | | |
| | thaliana] | | |
| 7 | Oryza sativa (japonica | DNA | NM_196346 |
| | cultivar-group) putative | | |
| | Cell elongation protein | | |
| | DIMINUTO (Cell elongation | | |
| | protein Dwarf1) | | |
| | (OSJNBb0058B20.18), mRNA | | |
| 8 | putative Cell elongation | AA | NP_921328 |
| | protein DIMINUTO (Cell | | |
| | elongation protein Dwarf1) | | |
| | [Oryza sativa (japonica | | |
| | cultivar-group)]. | | |
| 9 | Arabidopsis thaliana DWF1 | AA | |
| | protein V531D mutant | | |
| 10 | Arabidopsis thaliana DWF1 | AA | |
| | protein K521D, Y522G, R523D | | |
| | triple mutant | | |
| 11 | Arabidopsis thaliana DWF1 | AA | |
| | protein Δ518-539 mutant | | |
| 12 | Arabidopsis thaliana DWF1 | AA | |
| | protein V531D mutant-amino | | |
| | acids 518-539 | | |
| 13 | Arabidopsis thaliana DWF1 | AA | |
| | protein K521D, Y522G, R523D | | |
| | triple mutant-amino acids | | |
| | 518-539 | | |
| 14 | CPD (AT5G05690) | AA | AAM10042 |
| | cytochrome P450 90A1 | | |
| | [Arabidopsis thaliana]. | | |
| 15 | DWF4 (AT3G50660) | AA | NP_190635 |
| | DWF4 (DWARF 4) | | |
| | [Arabidopsis thaliana]. | | |
| 16 | DWF5 (AT1G50430) | AA | NP_175460 |
| | DWF5 (DWARF 5); sterol Δ7 | | |
| | reductase [Arabidopsis | | |
| | thaliana] | | |
| 17 | CPD (27 AA region) | AA | |
| 18 | N-DWF4 (25 AA region) | AA | |
| 19 | C-DWF4 (24 AA region) | AA | |
| 20 | DWF5 (25 AA region) | AA | |
| 21 | Arabidopsis thaliana wild- | AA | |
| | type CaM-binding peptide | | |
| 22 | DWF4 L21D mutant (25 amino | AA | |
| | acid segment) | | |
| 23 | DWF4 L28V mutant (25 amino | AA | |
| | acid segment) | | |

TABLE 1
Representative DWARF1 (DWF1) orthologues and representative
Accession Numbers (see FIG. 4a)

| | | Protein Accession |
|---|---|---|
| Title | Accession No. | No. |
| Homo sapiens (Hs) | NM_014762 | HPRD:HPRD_05916 |
| | NM_014762.2 | NIM:606418 |
| | GI:56790943 | |
| Pea. Zea mays brassino- | AY523572 | AAS90832.1 |
| steroid biosynthesis- | AY523572.1 | GI:46391568 |
| like proteins (DWF1) | GI:46391567 | |
| mRNA (Ps) | | |
| Arabidopsis thaliana | NM_180285 | NP_850616.1 |
| cell elongation | NM_180285.1 | GI:30685509 |
| protein (At) | GI:30685508 | |
| Arabidopsis thaliana | AY072216 | AAL60037.1 |
| putative cell | AY072216.1 | GI:18176398 |
| elongation protein | GI:18176397 | |
| (At) | | |
| Arabidopsis thaliana | NM_112872 | NP_188616.1 |
| cell elongation | NM_112872.2 | GI:15230955 |
| protein (At) | GI:30685505 | |
| Arabidopsis thaliana | AY096472 | AAM20112.1 |
| putative cell | AY096472.1 | GI:20465919 |
| elongation protein | GI:20465918 | |
| (At) | | |
| Oryza sativa mRNA | AJ277790 | CAC81901.1 |
| for cytochrome | AJ277790.1 | GI:47155289 |
| P450 Rice (Os) | GI:47155288 | |
| Oryza sativa | NM_196346 | NP_921328.1 |
| (japonica | NM_196346.1 | GI:37534052 |
| cultivar-group) | GI:37534051 | |
| putative cell | | |
| elongation protein | | |
| DIMINUTO Rice (Os) | | |
| Mus musculus 24- | NM_053272 | NP_444502.1 |
| dehydrocholesterol | NM_053272.1 | GI:16716609 |
| reductase Mouse | GI:16716608 | |
| (Mm) | | |
| Lycopersicon esculenrum | AY584532 | AAT90376.1 |
| DWARF1/DMINUTO | AY584532.1 | GI:50952901 |
| Tomato homolog of | GI:50952900 | |
| DIMINUTO/DWARF1 | | |

### PARTICULAR REFERENCES CITED

1. Clouse, S. D. Brassinosteroid signal transduction: clarifying the pathway from ligand perception to gene expression. Mol Cell 10, 973-82 (2002).
2. Nernhauser, J. L. & Chory, J. BRing it on: new insights into the mechanism of brassinosteroid action. J Exp Bot 55, 265-70 (2004).
3. Clouse, S. D. Integration of light and brassinosteroid signals in etiolated seedling growth. Trends Plant Sci 6, 443-5 (2001).
4. Snedden, W. A. & Fromm, H. Calmodulin as a versatile calcium signal transducer in plants. New Phytologist 151, 35-66 (2001).
5. Yang, T. & Poovaiah, B. W. Calcium/calmodulin-mediated signal network in plants. Trends Plant Sci 8, 505-12 (2003).
6. Klahre, U. et al. The Arabidopsis DIMINUTO/DWARF1 gene encodes a protein involved in steroid synthesis. Plant Cell 10, 1677-90 (1998).
7. Choe, S. et al. The Arabidopsis dwarf1 mutant is defective in the conversion of 24-methylenecholesterol to campesterol in brassinosteroid biosynthesis. Plant Physiol 119, 897-907 (1999).
8. Fujioka, S. & Yokota, T. Biosynthesis and Metabolism of Brassinosteroids. Annu Rev Plant Physiol Plant Mol Biol 54, 137-164 (2003).
9. Thummel, C. S. & Chory, J. Steroid signaling in plants and insects—common themes, different pathways. Genes Dev. 16, 3113-3129 (2002).
10. Wang, Z. Y., Seto, H., Fujioka, S., Yoshida, S. & Chory, J. BRI1 is a critical component of a plasma-membrane receptor for plant steroids. Nature 410, 380-3 (2001).
11. Li, J. et al. BAKI, an Arabidopsis LRR receptor-like protein kinase, interacts with BRI1 and modulates brassinosteroid signaling. Cell 110, 213-22 (2002).
12. Nam, K. H. & Li, J. BRI1/BAK1, a receptor kinase pair mediating brassinosteroid signaling. Cell 110, 203-12 (2002).
13. Li, J. & Nam, K. H. Regulation of brassinosteroid signaling by a GSK3/SHAGGY-like kinase. Science 295, 1299-301 (2002).
14. Kang, J. G. et al. Light and brassinosteroid signals are integrated via a dark-induced small G protein in etiolated seedling growth. Cell 105, 625-36 (2001).
15. Du, L. & Poovaiah, B. W. A Novel Family of Ca<sup>2+</sup>/Calmodulin-Binding Proteins Involved in Transcriptional Regulation: Interaction with fsh/Ring3 Class Transcription Activators. Plant Molecular Biology 54, 549-569 (2004).
16. Hall, D. B. & Struhl, K. The VP16 Activation Domain Interacts with Multiple Transcriptional Components as Determined by Protein-Protein Cross-linking in Vivo. J. Biol. Chem. 277, 46043-46050 (2002).
17. Poovaiah, B. W., Takezawa, D., An, G. & Han, T. J. Regulated expression of a calnodulin isoform alters growth and development in potato. J Plant Physiol 149, 553-8 (1996).
18. Zielinski, R. E. Characterization of three new members of the Arabidopsis thaliana calmodulin gene family: conserved and highly diverged members of the gene family functionally complement a yeast calmodulin null. Planta 214, 446-55 (2002).
19. Bouche, N., Scharlat, A., Snedden, W., Bouchez, D. & Fromm, H. A novel family of calmodulin-binding transcription activators in multicellular organisms. J Biol Chem 277, 21851-61 (2002).
20. Choe, S. et al. Lesions in the sterol 7 reductase gene of Arabidopsis cause dwarfism due to a block in brassinosteroid biosynthesis. The Plant Journal 21, 431-443 (2000).
21. CHORY, J. & LI, J. Gibberellins, brassinosteroids and light-regulated development. Plant, Cell & Environment 20, 801-806 (1997).
22. Szekeres, M. et al. Brassinosteroids rescue the deficiency of CYP90, a cytochrome P450, controlling cell elongation and de-etiolation in Arabidopsis. Cell 85, 171-82 (1996).
23. Alonso, J. M. et al. Genome-Wide Insertional Mutagenesis of Arabidopsis thaliana. Science 301, 653-657 (2003).
24. Li, J., Nagpal, P., Vitart, V., McMorris, T. C. & Chory, J. A Role for Brassinosteroids in Light-Dependent Development of Arabidopsis. Science 272, 398-401 (1996).
25. Shacklock, P. S., Read, N. D. & Trewavas, A. J. Cytosolic Free Calcium Mediates Red Light-Induced Photomorphogenesis. Nature 358, 753-755 (1992).
26. Baum, G., Long, J. C., Jenkins, G. I. & Trewavas, A. J. Stimulation of the blue light phototropic receptor NPH1 causes a transient increase in cytosolic Ca²⁺. PNAS 96, 13554-13559 (1999).
27. Schumacher, K. et al. The Arabidopsis det3 mutant reveals a central role for the vacuolar H+-ATPase in plant growth and development. Genes Dev. 13, 3259-3270 (1999).
28. Allen, G. J. et al. Alteration of Stimulus-Specific Guard Cell Calcium Oscillations and Stomatal Closing in Arabidopsis det3 Mutant. Science 289, 2338-2342 (2000).
29. Poovaiah, B. W. & Reddy, A. S. Calcium messenger system in plants. CRC Crit Rev Plant Sci 6, 47-103 (1987).

1. A method for modulating plant characteristics, comprising expressing within a plant of at least one brassinosteroid biosyntetic enzyme comprising at least one alteration of the amino acid sequence thereof, wherein the at least one amino acid sequence alteration modifies a cahnodulin-binding activity of the at least one enzyme, wherein at least one plant characteristic is modified.

2. The method of claim 1, wherein the at least one amino acid sequence alteration comprises an amino acid substitution, deletion or insertion.

3. The method of claim 2, wherein the at least one amino acid sequence alteration comprises an amino acid substitution.

4. The method of claim 3, wherein the at least one amino acid sequence alteration comprises a non-conservative amino acid substitution.

5. The method of claim 1, wherein, the at least one plant characteristic comprises at least one of size, growth, productivity, and fertility.

6. The method of claim 5, wherein the at least one plant characteristic comprises dwarfism or semi-dwarfism.

7. The method of claim 1, wherein the at least one alteration in amino acid sequence is in a calmodulin-binding domain of the at least one calmodulin-binding brassinosteroid biosyntetic enzyme.

8. The method of claim 1, wherein the at least one brassinosteroid biosyntetic enzyme comprises at least one selected from the group consisting of DWF1, DWF4, DWF5, CPD and plant orthologs thereof.

9. The method of claim 7, wherein the at least one brassinosteroid biosyntetic enzyme comprises at least one selected from the group consisting of DWF1, and calmodulin-binding orthologs thereof.

10. The method of claim 9, wherein the orthologs are plant orthologs.

11. The method of claim 1, wherein expressing comprises expressing within a plant of a plurality brassinosteroid biosyntetic enzymes, in each case comprising at least one alteration of the amino acid sequence thereof.

12. The method of claim 1, wherein expressing within a plant of at least one brassinosteroid biosyntetic enzyme comprising at least one alteration of the amino acid sequence thereof comprises said expressing in the presence of expression of a respective endogenous non-altered calmodulin-binding brassinosteroid biosyntetic enzyme.

13. The method of claim 1, wherein expressing within a plant of at least one brassinosteroid biosyntetic enzyme comprising at least one alteration of the amino acid sequence thereof comprises said expressing in the absence of, or in the presence of reduced expression of a respective endogenous non-altered calmodulin-binding brassinosteroid biosyntetic enzyme.

14. The method of claim 1, wherein expressing within a plant of at least one brassinosteroid biosyntetic enzyme comprising at least one alteration of the amino acid sequence thereof comprises said expressing in the presence of expression of a mutant or inactivated respective endogenous brassinosteroid biosyntetic enzyme that may or may not have calmodulin-binding acitity.

15. The method of claim 1, wherein the plant is a crop plant.

16. The method of claim 15, wherein the crop plant is Hops.

17. A plant generated by the method of claim 1.

18. The plant of claim 17, wherein the plant is a crop plant.

19. The plant of claim 18, wherein the crop plant is Hops.

20. A method for modulating the activity of a brassinosteroid biosyntetic enzyme, comprising introduction into the amino acid sequence of a calmodulin-binding brassinosteroid biosyntetic enzyme at least one amino acid sequence alteration, wherein the at least one amino acid sequence alteration modifies a calmodulin-binding activity of the at least one enzyme.

21. The method of claim 20, wherein the at least one amino acid sequence alteration comprises an amino acid substitution, deletion or insertion.

22. The method of claim 21, wherein the at least one amino acid sequence alteration comprises an amino acid substitution.

23. The method of claim 22, wherein the at least one amino acid sequence alteration comprises a non-conservative amino acid substitution.

24. The method of claim 20, wherein the at least one alteration in amino acid sequence is in a calmodulin-binding domain of the at least one calmodulin-binding brassinosteroid biosyntetic enzyme.

25. The method of claim 20, wherein the calmodulin-binding brassinosteroid biosyntetic enzyme is selected from the group consisting of DWF1, DWF4, DWF5, CPD and calmodulin-binding orthologs thereof.

26. The method of claim 25, wherein the ortholog is a plant ortholog.

27. The method of claim 25, wherein the at least one calmodulin-binding brassinosteroid biosyntetic enzyme comprises at least one selected from the group consisting of DWF1, and calmodulin-binding orthologs thereof.

28. The method of claim 27, wherein the ortholog is a plant ortholog.

29. An nucleic acid or expression vector encoding at least one brassinosteroid biosyntetic enzyme comprising at least one alteration of the amino acid sequence thereof, wherein the at least one amino acid sequence alteration modifies a calmodulin-binding activity of the at least one enzyme.

30. A cell transfected with a nucleic acid or expression vector encoding at least one brassinosteroid biosyntetic enzyme comprising at least one alteration of the amino acid sequence thereof, wherein the at least one amino acid sequence alteration modifies a calmodulin-binding activity of the at least one enzyme.

31. An isolated protein, comprising at least one alteration of the amino acid sequence of a brassinosteroid biosyntetic enzyme, wherein the at least one amino acid sequence alteration modifies a calmodulin-binding activity of the at least one enzyme.

32. The isolated protein of claim 31, wherein the brassinosteroid biosyntetic enzyme is selected from the group consisting of DWF1, DWF4, DWF5, CPD and calmodulin-binding orthologs thereof.

33. The isolated protein of claim 32, wherein the ortholog is a plant ortholog.

34. The isolated protein of claim 32, wherein the at least one brassinosteroid biosyntetic enzyme comprises at least one selected from the group consisting of DWF1, and calmodulin-binding orthologs thereof.

35. The isolated protein of claim 34, wherein the ortholog is a plant ortholog
